SEQUENCE LISTING (30 sequences)

SEQ ID NO: 1 - protein, 14 aa, FeLV
CAYCKEKGHWAKDC

SEQ ID NO: 2 - protein, 14 aa, R-MuLV
CAYCKEKGHWAKDC

SEQ ID NO: 3 - protein, 14 aa, M-MuLV
CAYCKEKGHWAKDC

SEQ ID NO: 4 - protein, 14 aa, Akv-MuLV
CAYCKEKGHWAKDC

SEQ ID NO: 5 - protein, 14 aa, BaEV
CAYCKERGHWTKDC

SEQ ID NO: 6 - protein, 43 aa, Artificial Sequence
  A synthetic peptide
  SITE (15)...(29): Xaa = any amino acid
CFKCGRKGHFAKNCXXXXXXXXXXXXXXXCPRCKRGKHWANEC

SEQ ID NO: 7 - protein, 41 aa, Artificial Sequence
  A synthetic peptide
  SITE (15)...(27): Xaa = any amino acid
CFSCGKTGHIKRDCXXXXXXXXXXXXXCPRCKKGYHWKSEC

SEQ ID NO: 8 - protein, 40 aa, Artificial Sequence
  A synthetic peptide
  SITE (15)...(26): Xaa = any amino acid
CYTCGSPGHYQAQCXXXXXXXXXXXXCELCNGMGHNAKQC

SEQ ID NO: 9 - protein, 39 aa, Artificial Sequence
  A synthetic peptide
  SITE (15)...(25): Xaa = any amino acid
CYRCLKEGHWARDCXXXXXXXXXXXCPICKDPSHWKRDC

SEQ ID NO: 10 - protein, 37 aa, Artificial Sequence
  A synthetic peptide
  SITE (15)...(23): Xaa = any amino acid
CFRCGKAGHWSRDCXXXXXXXXXCPLCQDPTHWKRDC

SEQ ID NO: 11 - protein, 35 aa, Artificial Sequence
  A synthetic peptide
  SITE (15)...(21): Xaa = any amino acid
CFNCGKEGHTARNCXXXXXXXCWKCGKEGHQMKDC

SEQ ID NO: 12 - protein, 35 aa, Artificial Sequence; identical to SEQ ID NO: 11
  A synthetic peptide
  SITE (15)...(21): Xaa = any amino acid

SEQ ID NO: 13 - protein, 35 aa, Artificial Sequence; identical to SEQ ID NO: 11
  A synthetic peptide
  ACT_SITE (15)...(21): Xaa = any amino acid

SEQ ID NO: 14 - protein, 35 aa, Artificial Sequence
  A synthetic peptide
  SITE (15)...(21): Xaa = any amino acid
CFNCGKEGHIARNCXXXXXXXCWKCGKEGHQMKDC

SEQ ID NO: 15 - protein, 35 aa, Artificial Sequence; identical to SEQ ID NO: 14
  A synthetic peptide
  ACT_SITE (15)...(21): Xaa = any amino acid

SEQ ID NO: 16 - protein, 35 aa, Artificial Sequence
  A synthetic peptide
  SITE (15)...(21): Xaa = any amino acid
CFNCGKEGHIAKNCXXXXXXXCWKCGKEGHQMKDC

SEQ ID NO: 17 - protein, 35 aa, Artificial Sequence
  A synthetic peptide
  SITE (15)...(21): Xaa = any amino acid
CFNCGKEGHIAKNCXXXXXXXCWRCGREGHQMKDC

SEQ ID NO: 18 - protein, 35 aa, Artificial Sequence
  A synthetic peptide
  ACT_SITE (15)...(21): Xaa = any amino acid
CFNCGKEGHIARNCXXXXXXXCWKCGREGHQMKDC

SEQ ID NO: 19 - protein, 35 aa, Artificial Sequence
  A synthetic peptide
  SITE (15)...(21): Xaa = any amino acid
CFNCGKVGHIAKNCXXXXXXXCWKCGKEGHQMKDC

SEQ ID NO: 20 - protein, 35 aa, Artificial Sequence
  A synthetic peptide
  SITE (15)...(21): Xaa = any amino acid
CFNCGKEGHLARNCXXXXXXXCWKCGKEGHQMKDC

SEQ ID NO: 21 - protein, 35 aa, Artificial Sequence
  A synthetic peptide
  SITE (15)...(21): Xaa = any amino acid
CFNCGKEGHIAKNCXXXXXXXCWRCGKEGHQLKDC

SEQ ID NO: 22 - protein, 35 aa, Artificial Sequence
  A synthetic peptide
  ACT_SITE (15)...(21): Xaa = any amino acid
CWNCGKEGHSARQCXXXXXXXCWKCGKPGHIMTNC

SEQ ID NO: 23 - protein, 35 aa, Artificial Sequence
  A synthetic peptide
  SITE (15)...(21): Xaa = any amino acid
CWNCGKEGHSARQWXXXXXXXCWKCGKSGHVMANC

SEQ ID NO: 24 - protein, 35 aa, Artificial Sequence
  A synthetic peptide
  SITE (15)...(21): Xaa = any amino acid
CWNCGKEGHSARQCXXXXXXXCWKCGKMDHVMAKC

SEQ ID NO: 25 - protein, 35 aa, Artificial Sequence; identical to SEQ ID NO: 24
  A synthetic peptide
  SITE (15)...(21): Xaa = any amino acid

SEQ ID NO: 26 - protein, 33 aa, Artificial Sequence
  A synthetic peptide
  ACT_SITE (15)...(19): Xaa = any amino acid
CYNCGKPGHLSSQCXXXXXCFKCKQPGHFSKQC

SEQ ID NO: 27 - protein, 33 aa, Artificial Sequence
  A synthetic peptide
  SITE (15)...(19): Xaa = any amino acid
CYNCGKPGHLARQCXXXXXCHHCGKRGHMQKDC

SEQ ID NO: 28 - protein, 14 aa, Artificial Sequence
  A synthetic peptide
  SITE 2, 3, 5, 6, 7, 8, 10, 11, 12, 13: Xaa = any amino acid
CXXCXXXXHXXXXC

SEQ ID NO: 29 - protein, 7 aa, Artificial Sequence
  A synthetic peptide
IQKGQAN

SEQ ID NO: 30 - protein, 10 aa, Artificial Sequence
  A synthetic peptide
IQKGQANFLG

(19) United States
(12) Patent Application Publication
(10) Pub. No.: US 2007/0099276 A1
(43) Pub. Date: May 3, 2007

(54) Retrovirus-like particles and retroviral vaccines

(76) Inventors: David Ott, Frederick, MD (US); Dexter Poon, Rockville, MD (US); Robert Gorelick, Rockville, MD (US)

Correspondence Address: SCHWEGMAN, LUNDBERG, WOESSNER & KLUTH/NIH, PO BOX 2938, MINNEAPOLIS, MN, 55402-0938, US

(21) Appl. No.: 11/413,614

(22) Filed: Apr. 27, 2006

Related U.S. Application Data
(63) Continuation of application No. PCT/US03/33897, filed on Oct. 25, 2003.

(51) Int. Cl. C12P 19/34; C12N 5/06; C12N 5/16

(52) U.S. Cl. 435/91.1; 435/345

(57) ABSTRACT

The invention relates to retrovirus-like particles having an altered nucleocapsid domain that inhibits packaging of genomic RNA into the retrovirus-like particle, and a protease having reduced enzymatic activity. The invention also provides nucleic acid constructs that encode the retrovirus-like particles. Also provided are immunological compositions, vaccines, and DNA vaccines containing the retrovirus-like particles and nucleic acid constructs that can be used to immunize a mammal against infection by a retrovirus. Methods to produce retrovirus-like particles are also provided.

### RELATED APPLICATIONS

This application is a continuation under 35 U.S.C. 111(a) of PCT/US2003/033897, filed Oct. 25, 2003 and published as WO 2005/051419 A1, filed Jun. 9, 2005, which application and publication are incorporated herein by reference and made a part hereof.

### GOVERNMENT FUNDING

The invention described herein was developed with the support of Federal funds from the National Cancer Institute, National Institutes of Health, under Contract No. NO1-CO-12400. The United States Government has certain rights in the invention.

### FIELD OF THE INVENTION

The invention relates to the field of immunology. Specifically, the invention relates to methods and materials that can be used to stimulate the immune system of a human or animal to produce an immune response against a retrovirus. More specifically, the invention relates to retrovirus-like particles that can be used to manufacture vaccines and immune compositions against infectious retroviruses.

### BACKGROUND OF THE INVENTION

Human immunodeficiency virus (HIV) proviral DNA, such as HIV type 1, includes three major genes, gag, pol, and env, as well as a number of accessory genes. Expression from the proviral long terminal repeat promoters ultimately leads to the transcription of spliced and full-length viral RNAs. The viral Gag protein is translated from the full-length messenger RNA and is synthesized initially as the polyprotein precursor Pr55^{gag}. Cellular expression of Pr55^{gag} has been shown to result in the formation of retrovirus-like particles. During or shortly after budding through the cellular membrane, Pr55^{gag} proteins are cleaved by the HIV protease (PR) encoded by the pol open reading frame, which yields a processing intermediate and the mature Gag proteins: matrix (MA), capsid (CA), nucleocapsid (NC), and p6. Two interdomain peptides p2 (between CA and NC) and p1 (between NC and p6) also are generated via this event.

The nucleocapsid protein acts in many diverse steps of retroviral assembly and infection. The nucleocapsid protein consists of an N-terminal, positively charged (basic) region, a proximal Cys-His finger motif, an interfinger region, a distal Cys-His finger motif, and a C-terminal section. The Cys-His finger motif has the following amino acid sequence: Cys-X₂-Cys-X₄-His-X₄-Cys (SEQ ID NO:28), wherein X represents variable amino acids. In the Gag precursor protein, the nucleocapsid zinc fingers are critical for specific genomic RNA packaging, as well as efficient reverse transcription and integration. The zinc fingers do not appear to be of primary importance for particle formation. Rather, the N-terminal basic amino acids appear to be required for general RNA binding and for proper Gag-Gag interaction.

Disruption of the nucleocapsid protein causes retrovirus-like particles to be produced that do not efficiently package genomic RNA. Due to this defect, the infectivity of these particles is severely reduced or completely eliminated. The retrovirus-like particles are therefore useful for the preparation of vaccines and other types of immunological compositions. These vaccines and immunological compositions can be used to immunize animals and humans against infection by a retrovirus corresponding to the retrovirus-like particle. For example, a retrovirus-like particle prepared from an altered human immunodeficiency virus type 1 (HIV-1) molecular clone can be used to immunize against infection by HIV-1.

Accordingly, what are needed are methods and nucleic acid constructs that can be used to efficiently produce retrovirus-like particles.

### SUMMARY OF THE INVENTION

These and other needs are met by the present invention. The present invention provides: retrovirus-like particles having an altered nucleocapsid domain that inhibits packaging of genomic RNA into the retrovirus-like particle, and a protease having reduced enzymatic activity; nucleic acid constructs that include a full-length proviral clone that encodes a retrovirus-like particle having an altered nucleocapsid domain that inhibits packaging of genomic RNA into the retrovirus-like particle, and a protease having reduced enzymatic activity when expressed in a host cell; a cell that includes a nucleic acid construct that encodes a retrovirus-like particle having an altered nucleocapsid domain that inhibits packaging of genomic RNA into the retrovirus-like particle, and a protease having reduced enzymatic activity; and a method to increase production of retrovirus-like particles that involves contacting a host cell that produces a retrovirus-like particle having an altered nucleocapsid domain that inhibits packaging of genomic RNA into the retrovirus-like particle with a retroviral protease inhibitor. The invention also provides a retrovirus-like particle produced according to the method of the invention; a method of producing a retrovirus-like particle having a deletion in the nucleocapsid region and an inactivated retroviral protease that includes expressing a nucleic acid construct encoding the retrovirus-like particle in a host cell for a sufficient time to produce the retrovirus-like particle, and isolating the retrovirus-like particle; and an immunogenic composition that includes an isolated retrovirus-like particle having an altered nucleocapsid domain that inhibits packaging of genomic RNA into the retrovirus-like particle, and a protease having reduced enzymatic activity, and a pharmaceutically acceptable carrier. The invention further provides a vaccine that includes a retrovirus-like particle having an altered nucleocapsid domain that inhibits packaging of genomic RNA into the retrovirus-like particle, an inactivated protease, and a pharmaceutical carrier; a DNA vaccine that includes a nucleic acid construct that encodes a retrovirus-like particle having an altered nucleocapsid domain that inhibits packaging of genomic RNA into the retrovirus-like particle, and a protease having reduced enzymatic activity, and a pharmaceutical carrier; a method to immunize a mammal against an infectious retrovirus comprising administering a therapeutically effective amount of an immune composition, a vaccine, or a DNA vaccine according to the invention to the mammal in need thereof; a non-infectious mutant of an infectious retrovirus that includes a form of the infectious retrovirus having a mutation or deletion in a nucleocapsid domain of the infectious retrovirus, and a mutation or deletion in a protease domain of the infectious retrovirus; and a kit that includes packaging material and an immune composition, a vaccine, or a DNA vaccine according to the invention.

The invention provides a retrovirus-like particle having an altered nucleocapsid domain that inhibits packaging of genomic RNA into the retrovirus-like particle, and a protease having reduced enzymatic activity.

Preferably the retrovirus-like particle is derived by mutating another retrovirus-like particle. More preferably the retrovirus-like particle is derived by mutating a non-infectious retrovirus. Even more preferably the retrovirus-like particle is derived by mutating an infectious retrovirus. Most preferably the retrovirus-like particle is derived by mutating a human immunodeficiency virus. Preferably the nucleocapsid domain includes a cysteine array having the sequence -Cys-X₂-Cys-X₄-His-X₄-Cys- (SEQ ID NO:28), where X represents a variable amino acid. Preferably the nucleocapsid domain is mutated by exchanging at least one amino acid for another amino acid in the nucleocapsid domain. More preferably the nucleocapsid domain is mutated by inserting at least one amino acid into the nucleocapsid domain. Still more preferably the nucleocapsid domain is mutated by deleting at least one amino acid from the nucleocapsid domain. Yet still more preferably the nucleocapsid domain is mutated by exchanging a variable amino acid for another amino acid in the cysteine array. Even yet still more preferably the nucleocapsid domain is mutated by exchanging one or more cysteines or a histidine for another amino acid in the cysteine array. Still even yet still more preferably the nucleocapsid domain is mutated by deleting one or more cysteines or a histidine in the cysteine array. Yet still even yet still more preferably the nucleocapsid domain is mutated by deleting the cysteine array. Most preferably the nucleocapsid domain is mutated by deleting the nucleocapsid domain.

Preferably the protease is from human immunodeficiency virus. Preferably the protease is mutated by inserting at least one amino acid into the protease. More preferably the protease is mutated by deleting at least one amino acid from the protease. Even more preferably the protease is mutated by exchanging at least one amino acid for another amino acid in the protease. Still even more preferably the protease is mutated by exchanging the arginine at amino acid position 57 to another amino acid. Yet still even more preferably the protease is mutated by exchanging the arginine at amino acid position 57 to glycine. Even yet still even more preferably the protease is mutated by exchanging the aspartic acid at amino acid position 25 to another amino acid. Most preferably the protease is mutated by exchanging aspartic acid at amino acid position 25 to asparagine. Preferably the protease activity of the retrovirus-like particle is reduced by at least one order of magnitude when compared to the activity of the protease from the wild-type virus from which the retrovirus-like particle was derived. More preferably the protease activity of the retrovirus-like particle is reduced by at least three orders of magnitude when compared to the activity of the protease from the wild-type virus from which the retrovirus-like particle was derived. Most preferably the protease activity of the retrovirus-like particle is reduced by at least five orders of magnitude when compared to the activity of the protease from the wild-type virus from which the retrovirus-like particle was derived.

Preferably the infectivity of the retrovirus-like particle is reduced by at least three orders of magnitude when compared to the infectivity to the wild-type virus from which the retrovirus-like particle was derived. More preferably the infectivity of the retrovirus-like particle is reduced by at least five orders of magnitude when compared to the infectivity to the wild-type virus from which the retrovirus-like particle was derived. Even more preferably the infectivity of the retrovirus-like particle is reduced by at least eight orders of magnitude when compared to the infectivity to the wild-type virus from which the retrovirus-like particle was derived. Most preferably the retrovirus-like particle is non-infective.

Preferably the genomic RNA content of the retrovirus-like particle is reduced when compared to the genomic RNA content of the wild-type virus from which the retrovirus-like particle was derived. More preferably the genomic RNA content of the retrovirus-like particle is reduced by at least one order of magnitude when compared to the genomic RNA content of the wild-type virus from which the retrovirus-like particle was derived. Even more preferably the genomic RNA content of the retrovirus-like particle is reduced by at least three orders of magnitude when compared to the genomic RNA content of the wild-type virus from which the retrovirus-like particle was derived. Still even more preferably the genomic RNA content of the retrovirus-like particle is reduced by at least five orders of magnitude when compared to the genomic RNA content of the wild-type virus from which the retrovirus-like particle was derived. Most preferably genomic RNA is not detectable in the retrovirus-like particle.

Preferably the retrovirus-like particle is derived from equine infectious anemia virus. More preferably the retrovirus-like particle is derived from bovine leukemia virus. Even more preferably the retrovirus-like particle is derived from feline leukemia virus. Still even more preferably the retrovirus-like particle is derived from baboon endogenous virus. Yet still even more preferably the retrovirus-like particle is derived from simian SRV-1 retrovirus. Even yet still even more preferably the retrovirus-like particle is derived from simian immunodeficiency virus. Still even yet still even more preferably the retrovirus-like particle is derived from human immunodeficiency virus type 2. Most preferably the retrovirus-like particle is derived from human immunodeficiency virus type 1.

The invention provides a nucleic acid construct that include a proviral clone that encodes a retrovirus-like particle having an altered nucleocapsid domain that inhibits packaging of genomic RNA into the retrovirus-like particle, and a protease having reduced enzymatic activity.

Preferably the nucleic acid construct is derived by mutating another nucleic construct that encodes a retrovirus-like particle. More preferably the nucleic acid construct is derived by mutating a nucleic construct that encodes a non-infectious retrovirus. Even more preferably the nucleic acid construct is derived by mutating a nucleic construct that encodes an infectious retrovirus. Most preferably the nucleic acid construct is derived by mutating a nucleic acid construct that encodes human immunodeficiency virus.

Preferably the nucleic acid construct encodes a nucleocapsid domain that includes a cysteine array having the sequence -Cys-X₂-Cys-X₄-His-X₄-Cys- (SEQ ID NO:28), where X represents a variable amino acid. Preferably the nucleic acid construct is mutated so that it encodes a nucleocapsid domain having at least one amino acid exchanged for another amino acid. More preferably the nucleic acid construct is mutated so that it encodes a nucleocapsid domain having at least one amino acid inserted into the nucleocapsid domain. Still more preferably the nucleic acid construct is mutated so that it encodes a nucleocapsid domain having at least one amino acid deleted from the nucleocapsid domain. Yet still more preferably the nucleic acid construct is mutated so that it encodes a nucleocapsid domain having at least one variable amino acid exchanged for another amino acid in the cysteine array. Even yet still more preferably the nucleic acid construct is mutated so that it encodes a nucleocapsid domain having one or more cysteines or a histidine exchanged for another amino acid in the cysteine array. Still even yet still more preferably the nucleic acid construct is mutated so that it encodes a nucleocapsid domain having one or more cysteines or a histidine deleted in the cysteine array. Yet still even yet still more preferably the nucleic acid construct is mutated so that the cysteine array is deleted from the nucleocapsid domain. Most preferably the nucleic acid construct is mutated so that the nucleocapsid domain is deleted from the retrovirus-like particle.

Preferably the protease is from human immunodeficiency virus. Preferably the nucleic acid construct is mutated so that it encodes a protease having at least one amino acid inserted into the protease. More preferably the nucleic acid construct is mutated so that it encodes a protease having at least one amino acid deleted from the protease. Even more preferably the nucleic acid construct is mutated so that it encodes a protease having at least one amino acid exchanged for another amino acid in the protease. Still even more preferably the nucleic acid construct is mutated so that it encodes a protease having the arginine at amino acid position 57 exchanged with another amino acid. Yet still even more preferably the nucleic acid construct is mutated so that it encodes a protease having the arginine at amino acid position 57 exchanged with glycine. Even yet still even more preferably the nucleic acid construct is mutated so that it encodes a protease having the aspartic acid at amino acid position 25 exchanged with another amino acid. Most preferably the nucleic acid construct is mutated so that it encodes a protease having the aspartic acid at amino acid position 25 exchanged with asparagine. Preferably the protease activity encoded by the nucleic acid construct is reduced by at least one order of magnitude when compared to the activity of the protease from the virion or virus-like particle from which the nucleic acid construct was derived. Preferably the protease activity encoded by the nucleic acid construct is reduced by at least three orders of magnitude when compared to the activity of the protease from the virion or virus-like particle from which the nucleic acid construct was derived. Preferably the protease activity encoded by the nucleic acid construct is reduced by at least five orders of magnitude when compared to the activity of the protease from the virion or virus-like particle from which the nucleic acid construct was derived.

Preferably the infectivity of the retrovirus-like particle encoded by the nucleic acid construct is reduced by at least three orders of magnitude when compared to the infectivity to the wild-type virus from which the retrovirus-like particle was derived. More preferably the infectivity of the retrovirus-like particle encoded by the nucleic acid construct is reduced by at least five orders of magnitude when compared to the infectivity to the wild-type virus from which the retrovirus-like particle was derived. Even more preferably the infectivity of the retrovirus-like particle encoded by the nucleic acid construct is reduced by at least eight orders of magnitude when compared to the infectivity to the wild-type virus from which the retrovirus-like particle was derived. Most preferably the retrovirus-like particle encoded by the nucleic acid construct is non-infective.

Preferably the genomic RNA content of the retrovirus-like particle encoded by the nucleic acid construct is reduced when compared to the genomic RNA content of the wild-type virus from which the retrovirus-like particle was derived. More preferably the genomic RNA content of the retrovirus-like particle encoded by the nucleic acid construct is reduced by at least one order of magnitude when compared to the genomic RNA content of the wild-type virus from which the retrovirus-like particle was derived. Even more preferably the genomic RNA content of the retrovirus-like particle encoded by the nucleic acid construct is reduced by at least three orders of magnitude when compared to the genomic RNA content of the wild-type virus from which the retrovirus-like particle was derived. Still even more preferably the genomic RNA content of the retrovirus-like particle encoded by the nucleic acid construct is reduced by at least five orders of magnitude when compared to the genomic RNA content of the wild-type virus from which the retrovirus-like particle was derived. Most preferably the genomic RNA content of the retrovirus-like particle encoded by the nucleic acid construct is not detectable in the retrovirus-like particle.

Preferably the nucleic acid construct that encodes the retrovirus-like particle is derived from equine infectious anemia virus. More preferably the nucleic acid construct that encodes the retrovirus-like particle is derived from bovine leukemia virus. Even more preferably the nucleic acid construct that encodes the retrovirus-like particle is derived from feline leukemia virus. Still even more preferably the nucleic acid construct that encodes the retrovirus-like particle is derived from baboon endogenous virus. Yet still even more preferably the nucleic acid construct that encodes the retrovirus-like particle is derived from simian SRV-1 retrovirus. Even yet still even more preferably the nucleic acid construct that encodes the retrovirus-like particle is derived from simian immunodeficiency virus. Still even yet still even more preferably the nucleic acid construct that encodes the retrovirus-like particle is derived from human immunodeficiency virus type 2. Most preferably the nucleic acid construct that encodes the retrovirus-like particle is derived from human immunodeficiency virus type 1.

The invention provides a cell that includes a nucleic acid construct of the invention. Preferably the cell is a prokaryotic cell. More preferably the cell is a eukaryotic cell. Preferably the prokaryotic cell is a bacterium. Preferably the eukaryotic cell is a yeast cell. More preferably the eukaryotic cell is a mammalian cell. Even more preferably the eukaryotic cell is a simian cell. Still even more preferably the eukaryotic cell is a human cell. Still even more preferably the eukaryotic cell is a human kidney cell. Yet still even more preferably the eukaryotic cell is a human adenocarcinoma cell. Most preferably the eukaryotic cell is a human T-cell leukemia cell.

The invention provides a method to increase production of retrovirus-like particles having an altered nucleocapsid domain that inhibits packaging of genomic RNA into the retrovirus-like particles that involves contacting a host cell that produces the retrovirus-like particles with a retroviral protease inhibitor. Preferably the protease inhibitor is a peptide. More preferably the protease inhibitor is a peptidomimetic. Even more preferably the protease inhibitor is saquinavir. Preferably the retrovirus-like particle is derived from equine infectious anemia virus. More preferably the retrovirus-like particle is derived from bovine leukemia virus. Even more preferably the retrovirus-like particle is derived from feline leukemia virus. Still even more preferably the retrovirus-like particle is derived from baboon endogenous virus. Yet still even more preferably the retrovirus-like particle is derived from simian SRV-1 retrovirus. Even yet still even more preferably the retrovirus-like particle is derived from simian immunodeficiency virus. Still even yet still even more preferably the retrovirus-like particle is derived from human immunodeficiency virus type 2. Most preferably the retrovirus-like particle is derived from human immunodeficiency virus type 1. Also provided by the invention is a retrovirus-like particle produced according to the method of the invention.

The invention provides a method of producing a retrovirus-like particle having a deletion in the nucleocapsid region and an inactivated retroviral protease that includes expressing a nucleic acid construct encoding the retrovirus-like particle in a host cell for a sufficient time to produce the retrovirus-like particle, and isolating the retrovirus-like particle.

The invention provides an immunogenic composition that includes an isolated retrovirus-like particle of the invention, and a pharmaceutically acceptable carrier. Preferably the immunogenic composition includes an adjuvant. Preferably the immunogenic composition is formulated in unit dosage form.

The invention provides a vaccine that includes a retrovirus-like particle of the invention, and a pharmaceutical carrier. Preferably the vaccine includes an adjuvant. Preferably the immunogenic composition is formulated in unit dosage form.

The invention provides a DNA vaccine that includes a nucleic acid construct of the invention, and a pharmaceutical carrier. Preferably the DNA vaccine is formulated in unit dosage form. Preferably the DNA vaccine includes a myonecrotic agent. Preferably the myonecrotic agent is bupivicaine. More preferably the myonecrotic agent is cardiotoxin. Preferably the DNA vaccine includes an adjuvant.

The invention provides a method to immunize a mammal against an infectious retrovirus comprising administering a therapeutically effective amount of an immune composition, a vaccine, or a DNA vaccine according to the invention to the mammal in need thereof. Preferably the mammal is a cat. More preferably the mammal is a horse. Even more preferably the mammal is a cow. Still even more preferably the mammal is a baboon. Yet still even more preferably the mammal is a monkey. Most preferably the mammal is a human.

The invention provides a non-infectious mutant of an infectious retrovirus that includes a form of the infectious retrovirus having a mutation or deletion in a nucleocapsid domain of the infectious retrovirus, and a mutation or deletion in a protease domain of the infectious retrovirus. Preferably the infectious retrovirus is equine infectious anemia virus. More preferably the infectious retrovirus is bovine leukemia virus. Even more preferably the infectious retrovirus is feline leukemia virus. Still even more preferably the infectious retrovirus is baboon endogenous virus. Yet still even more preferably the infectious retrovirus is simian SRV-1 retrovirus. Even yet still even more preferably the infectious retrovirus is simian immunodeficiency virus. Still even yet still even more preferably the infectious retrovirus is human immunodeficiency virus type 2. Most preferably the infectious retrovirus is human immunodeficiency virus type 1.

The invention provides a kit that includes packaging material and an immune composition, a vaccine, or a DNA vaccine according to the invention.

### BRIEF DESCRIPTION OF THE FIGURES

FIG. 1 illustrates diagrams of the Gag regions of the wild-type (NL4-3) and nucleocapsid deletion (DelNC) constructs. The nucleocapsid (NC) sequences remaining after deletion of the majority of the NC region are displayed in single amino acid code just below the diagram with the amino acid positions relative to NC at the fusion point indicated (SEQ ID NO:29).

FIGS. 2A-2F illustrate immunoblots of nucleocapsid deletion (DelNC) retrovirus-like particles produced by transfection. The antiserum or antibody used is indicated above the respective blots and samples are identified above the respective lanes (sssDNA=a virus preparation produced from transfected sheared salmon sperm DNA). Molecular masses, as calculated by relative mobility, and identities of bands are indicated at the margins of the blots.

FIGS. 3A-3B show high pressure liquid chromatography (HPLC) analysis of nucleocapsid deletion (DelNC) retrovirus-like particles. FIG. 3A shows chromatograms of equal percentages of NL4-3 virus and DelNC retrovirus-like particle preparations. Peaks containing Gag proteins are identified above the respective peaks. FIG. 3B shows protein sequence analysis of a portion of fraction 15 which is displayed at the top with the deleted nucleocapsid amino acids underlined (SEQ ID NO:30). Matrix assisted laser desorption/ionization (MALDI) technique mass spectrometry results of fraction 15 is presented at the bottom.

FIG. 4A shows a northern blot analysis of retrovirus-like particle and virion genomic RNA preparations. Samples contained 5.2×10⁶ cpm of reverse transcriptase activity from each mutant and wild-type virus preparation. The blot was probed with a ³²P-labaled 8.1 kbp AvaI fragment from pNL4-3. In addition to the wild-type NL4-3 sample (WT), 1/5 (WT/5), 1/25 (WT/25), and 1/125 (WT/125) dilutions of this sample were also were also tested. Samples are identified above their respective lanes with RNA markers indicated on the left and the size of the full-length genome indicated on the right.

FIG. 4B shows an autoradiogram of ³²P metabolically labeled retrovirus-like particles and virions as analyzed by RNA denaturing agarose gel electrophoresis. Virion and retrovirus-like particle preparations isolated from equal amounts of transfection culture supernatants were examined. Samples are indicated at the top of the gel, RNA marker sizes are indicated on the left, and the positions of the 9.3 kb viral RNA, and 28S and 18S ribosomal bands are indicated on the right. For both panels, “(−)-Control” denotes a virus preparation produced from transfected sheared salmon sperm DNA.

FIGS. 5A-5C show the result of equilibrium density gradient centrifugation of viruses and retrovirus-like particles. Profiles of wild-type NL4-3, DelNC and DelNC/PR_{R57G} viruses and retrovirus-like particles that were subjected to centrifugation through 10-50% (wt/vol) sucrose gradients are presented. The amount of retrovirus-like particles or virions detected as measured by RT activity (³H-TMP incorporation) or by scanning densitometry (pixel grayscale density) from a p6^{Gag} immunoblot of selected fractions is reported on the y-axis versus density in grams/milliliter of sucrose on the x-axis. Virions and retrovirus-like particles analyzed are identified to the left of the respective graphs.

FIGS. 6A-6D show electron micrographs of virions and retrovirus-like particles. Transmission electron micrographs of positively stained wild-type (FIG. 6A), DelNC (FIG. 6B), PR_{R57G} (FIG. 6C) and DelNC/PR_{R57G} (FIG. 6D) virion and retrovirus-like particle preparations are presented at 40,000× magnification. Enlarged images of representative virions and retrovirus-like particles present in this field and others are displayed underneath the field micrographs.

FIG. 7A shows immunoblots of retrovirus-like particle and virion preparations produced from transfection of cells with various constructs. The antiserum or antibody used is indicated above the respective blots and the samples analyzed are identified above the respective lanes (sssDNA=a virus preparation produced from transfected sheared salmon sperm DNA). Molecular masses, as calculated by relative mobility, and identities of bands are indicated at the margins of the blots.

FIG. 7B shows immunoblots of retrovirus-like particle and virion preparations produced from transfection of cells with various constructs in the presence or absence of 10 nM Saquinavir. The antiserum or antibody used is indicated above the respective blots and the samples analyzed are identified above the respective lanes (sssDNA=a virus preparation produced from transfected sheared salmon sperm DNA). Molecular masses, as calculated by relative mobility, and identities of bands are indicated at the margins of the blots. The addition of Saquinavir in the production of virus samples is indicated as PI “+” above the appropriate lanes. WT=wild-type.

### DETAILED DESCRIPTION OF THE INVENTION

The invention relates to the discovery that inhibition or elimination of retroviral protease activity, such as HIV-1 protease activity, increases production of retrovirus-like particles in which a portion of the nucleocapsid domain of the Gag polyprotein has been mutated or deleted. This increase in production is thought to result from increased budding efficiency of the retrovirus-like particles from the cells in which the retrovirus-like particles are assembled. The infectivity of these retrovirus-like particles is severely reduced or is eliminated. The retrovirus-like particles are also devoid of genomic RNA, or display severely reduced genomic RNA content when compared to wild-type virions. The invention also relates to a full length retroviral constructs, such as an HIV-1 DNA construct, that encodes a Gag polyprotein in which the nucleocapsid domain is mutated or deleted, and in which the retroviral protease is inactivated. Additionally, the invention relates to the use of retroviral protease inhibitors to increase production of retrovirus-like particles in which the nucleocapsid domain is mutated or deleted, and that contain a severely reduced quantity of genomic RNA.

Accordingly, retrovirus-like particles in which the nucleocapsid domain is mutated or deleted, and in which the retroviral protease is inactivated, may be used in the preparation of vaccines against numerous retroviruses that infect animals and humans, and in the preparation of diagnostic reagents for the retroviruses.

Definitions:

The term “altered nucleocapsid domain” refers to a nucleocapsid domain that has a mutation, insertion, or deletion in the nucleocapsid domain that inhibits packaging of genomic RNA into a retrovirus-like particle formed in part from the altered nucleocapsid. An insertion or deletion in the nucleocapsid domain may be the insertion or deletion of a single amino acid, or multiple amino acids into or from the nucleocapsid domain. For example, a deletion may be elimination of a histidine, elimination of one or more cysteine residues, or elimination of a histidine and one or more cysteine residues in the cysteine array found in the nucleocapsid domain. A deletion may also eliminate all or a large portion or the nucleocapsid domain. For example a deletion may be elimination of amino acids at positions 5-52 of the nucleocapsid domain as encoded by the infectious molecular clone pNL4-3 (Accession number AF324493). In another example, a deletion may be elimination of amino acids at positions 1-55 of the nucleocapsid domain as encoded by the infectious molecular clone pNL4-3 (Accession number AF324493). A mutation in an altered nucleocapsid can be exchange of one or more amino acids in the nucleocapsid domain with one or more other amino acids. For example, a cysteine or histidine residue in the cysteine array of the nucleocapsid may be exchanged with another amino acid.

The term “altered protease” refers to a retroviral protease having a mutation, insertion, or deletion that reduces or eliminates protease activity. A deletion in the retroviral protease may be deletion of a single amino acid, or elimination of multiple amino acids from the retroviral protease. A deletion may also eliminate all or a large portion or the retroviral protease. For example, deletion of the retroviral protease may be achieved by inserting a stop codon into the pol gene at the fourth amino acid position of the retroviral protease as described herein. A mutation in an altered retroviral protease can be the exchange of one or more amino acids in the retroviral protease for another amino acid. For example, the arginine at amino acid position 57 can be exchanged with another amino acid, such as glycine, to eliminate the protease activity of the retroviral protease. Insertion of one or more amino acids into the retroviral protease may be used to disrupt the active site of the enzyme and thereby reduce or eliminate the enzymatic activity of the protease.

An “adjuvant” is generally defined as a substance that nonspecifically enhances the immune response to an antigen. A variety of adjuvants may be employed with the immune compositions and vaccines of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, interleukin-7, or interleukin-12, may also be used as adjuvants.

The term “derived” refers to a retroviral virion or retrovirus-like particle which is mutated to produce a retrovirus-like particle of the invention having an altered nucleocapsid domain and an altered retroviral protease. For example, the nucleocapsid and protease of an infectious HIV-1 clone can be mutated to produce an HIV-1 retrovirus-like particle of the invention.

The term “nucleocapsid domain” refers to the nucleocapsid protein encoded by the gag gene of a retrovirus. For example, the pNL4-3 infectious molecular clone of HIV-1 encodes the p7^{NC} nucleocapsid protein.

I. Retrovirus-Like Particles

It has been discovered that inhibition or elimination of retroviral protease activity increases budding of retrovirus-like particles having mutations or deletions in the nucleocapsid domain from cells in which the retrovirus-like particles are assembled. Accordingly, the invention provides retrovirus-like particles having an altered nucleocapsid domain that inhibits packaging of genomic RNA into the retrovirus-like particle, and a retroviral protease having reduced or undetectable enzymatic activity. Combining an altered nucleocapsid domain with an inactive retroviral protease provides for the efficient production of retrovirus-like particles that can be used in the manufacture of vaccines and immunological compositions.

In addition, retrovirus-like particles having mutations at two sites increases the safety of the retrovirus-like particles for production of vaccines or immunological compositions because mutation at two sites severely reduces reversion of the retrovirus-like particle to a wild-type infectious virion. Accordingly, retrovirus-like particles of the invention are superior to retrovirus-like particles that were prepared prior to the invention.

Retrovirus-like particles of the invention can be prepared by altering the nucleocapsid domain, and the protease of numerous infectious retroviruses. Examples of infectious retroviruses that can be altered to produce retrovirus-like particles of the invention are presented below in Table I and have been further described (U.S. Pat. No. 5,674,720; and Fields Virology, 4th edit., Lippincott Williams & Wilkins, 2001).

The retroviral nucleocapsid domain may be altered by exchanging one or more amino acids with others such that the altered nucleocapsid causes reduced packaging of genomic RNA into the retrovirus-like particle, as compared to the amount of genomic RNA packaged into a wild-type virion. These amino acid exchanges may occur throughout the nucleocapsid domain. For example, exchange of an amino acid found in the cysteine array of the nucleocapsid domain can produce retrovirus-like particles that are profoundly deficient in their ability to package genomic RNA into retrovirus-like particles. Accordingly, retrovirus-like particles that are deficient in the packaging of genomic RNA can be made by exchanging a cysteine or histidine residue within the cysteine array with another amino acid. A variable amino acid within the cysteine array may also be exchanged for another amino acid to produce retrovirus-like particles that are deficient in the packaging of genomic RNA. Single amino acids, or multiple amino acids, may be exchanged in the nucleocapsid domain to produce retrovirus-like particles that are deficient in the packaging of genomic RNA.

The retroviral nucleocapsid domain may be altered by deleting one or more amino acids from the nucleocapsid domain such that the altered nucleocapsid causes reduced packaging of genomic RNA into the retrovirus-like particle, as compared to the amount of genomic RNA packaged into a wild-type virion. These amino acid deletions may occur throughout the nucleocapsid domain. For example, deletion of an amino acid found in the cysteine array of the nucleocapsid domain can produce retrovirus-like particles that are profoundly deficient in their ability to package genomic RNA into retrovirus-like particles. Accordingly, retrovirus-like particles that are deficient in the packaging of genomic RNA can be made by deleting a cysteine or histidine residue within the cysteine array. A variable amino acid within the cysteine array may also be deleted to produce retrovirus-like particles that are deficient in the packaging of genomic RNA. Single amino acids, or multiple amino acids, may be deleted from the nucleocapsid domain to produce retrovirus-like particles that are deficient in the packaging of genomic RNA.

The invention also provides retrovirus-like particles in which a large portion, or all, of the nucleocapsid domain is deleted. For example, amino acids at residue positions 5-52 of the nucleocapsid domain encoded by the pNL4-3 infectious molecular clone of HIV-1 can be deleted to produce a retrovirus-like particle that is deficient in the packaging of genomic RNA. Other examples of deletions of the nucleocapsid domain encoded by the pNL4-3 infectious molecular clone of HIV-1 include deletion of amino acids at residue positions 1-10, 1-20, 1-30, 1-40, 1-50, 1-55, 50-55, 40-50, 30-40, 20-30, 10-20, 10-50, 20-40, and the like.

The retroviral nucleocapsid domain may be altered by inserting one or more amino acids into the nucleocapsid domain such that the altered nucleocapsid causes reduced packaging of genomic RNA into the retrovirus-like particle, as compared to the amount of genomic RNA packaged into a wild-type virion. These amino acids insertions may occur throughout the nucleocapsid domain. For example, insertion of one or more amino acids into the cysteine array of the nucleocapsid domain can disrupt proper binding of genomic RNA by the nucleocapsid and produce retrovirus-like particles that are profoundly deficient in their ability to package genomic RNA into retrovirus-like particles. Single amino acids, or multiple amino acids, may be inserted into the nucleocapsid domain to produce retrovirus-like particles that are deficient in the packaging of genomic RNA.

A retrovirus-like particle of the invention has an altered retroviral protease having reduced or undetectable enzymatic activity, as well as an altered nucleocapsid domain that inhibits packaging of genomic RNA into the retrovirus-like particle. The pol gene encoding a retroviral protease can be mutated so that it expresses an altered retroviral protease having reduced or eliminated protease activity. Guidance as to where retroviral proteases can be mutated to reduce or eliminate their activity is known in the art (Loeb et al., Nature, 340:397 (1989); Swanstrom, Curr. Opin. Biotechnol., 5:409 (1994); Miller, Science, 246:1149 (1989); Kohl, Proc. Natl. Acad. Sci. USA, 85:4686 (1988); Beck et al., Curr. Drug Targets Infect. Disord., 2:37 (2002)). For example, mutagenesis studies of the HIV-1 protease revealed that the protease has three noncontiguous regions that have high mutational sensitivity. The first region includes amino acids residues between positions 22 and 33 that form a loop which runs along the presumed substrate binding cleft. This region includes the sequence ²⁵DTG²⁷ which has strong similarity to the active site of aspartic proteinases, where D25 is equivalent to the catalytically active aspartic acid residue (Toh et al., EMBO J. 4:1267 (1985)). The second region is a short glycine-rich sequence, ⁴⁷IGGIGG⁵², in the center of the protease sequence. The third region is the largest, T74 to R87, and is generally hydrophobic. These regions may be mutated to produce retroviral proteases having reduced enzymatic activity, or that lack enzymatic activity. For example, mutation of the aspartic acid at amino acid residue position 25 to asparagine (D25N) produces a retroviral protease that lacks enzymatic activity (Kohl, Proc. Natl. Acad. Sci. USA, 85:4686 (1988)). This aspartic acid is strictly conserved among retroviral proteases and can therefore be exchanged with another amino acid in other retroviral proteases to inactivate the other proteases. In another example, the arginine at amino acid position 57 can be exchanged with another amino acid, such as glycine, to eliminate the protease activity of the retroviral protease encoded by the pNL4-3 infectious molecular clone of HIV-1. A retroviral protease can also be altered by deletion of a single amino acid, or elimination of multiple amino acids from the retroviral protease. Amino acids can also be inserted into a retroviral protease to reduce or eliminate enzymatic activity.

Retrovirus-like particles of the invention can be created from other infectious viruses by altering the retroviral protease to have reduced activity, and making the corresponding mutations and deletions in the nucleocapsid domain of the infectious virus. Examples of numerous retroviruses are provided in Table I

II. Nucleic Acid Constructs

The invention provides nucleic acid constructs that are expressed within cells into which the nucleic acid constructs are inserted, and which cause the cells to produce retrovirus-like particles of the invention. A nucleic acid construct of the invention encodes an altered retroviral protease having reduced enzymatic activity, as well as an altered nucleocapsid domain that inhibits packaging of genomic RNA into the retrovirus-like particle. Nucleic acid constructs of the invention include, but are not limited to, those that cause a cell to produce any of the retrovirus-like particles described hereinabove when the nucleic acid construct is expressed with a cell.

A nucleic acid construct of the invention can be derived by altering numerous retroviral clones that can be infectious or non-infectious, provided that a virion or a retrovirus-like particle is produced when the retroviral clone is expressed within a cell. Many retroviral clones are known and can be modified through use of standard techniques to produce a nucleic acid construct of the invention. Examples of such retroviral clones have the following GenBank accession numbers: M18247 (Feline leukemia virus), NC_{—}001362 (Friend murine leukemia virus), D10032 (Baboon endogenous virus), M11841 (Simian SRV-1 type D retrovirus), NC_{—}001407 (Rous sarcoma virus), AF257515 (Bovine leukemia virus), AF327878 (Equine infectious anemia virus), NC_{—}001452 (Visna virus), AY159322 (Simian immunodeficiency virus), and AF324493 (Human immunodeficiency virus type 1 (HIV-1)), M31113 (Human immunodeficiency virus type 2 (HIV-2)), M15390 (Human immunodeficiency virus type 2, isolate ROD), J04498 (Human immunodeficiency virus type 2, isolate SBLISY), L07625 (Human immunodeficiency virus type 2 from strain HIV-2UC1), U22047 (Human immunodeficiency virus type 2).

The nucleocapsid and protease encoded by these retroviral clones can be altered to produce a nucleic acid construct of the invention through use of standard methods (Sambrook et al., Molecular Cloning: A Laboratory Manual, 3rd edition, Cold Spring Harbor Press, Cold Spring Harbor, N.Y. (2001)). For example, the nucleocapsid and protease encoded by the retroviral clones may be altered to encode amino acid exchanges, deletions, and insertions. Methods for mutagenesis and nucleotide sequence alterations are well known in the art. See, for example, Kunkel, Proc. Natl. Acad. Sci. USA, 82, 488 (1985); Kunkel et al., Methods in Enzymol., 154:367 (1987); U.S. Pat. No. 4,873,192; Walker and Gaastra, eds., Techniques in Molecular biology, MacMillan Publishing Company, New York (1983) and the references cited therein. The polymerase chain reaction may be used to alter the retroviral clones. Known methods of polymerase chain reaction “PCR” include, but are not limited to, methods using paired primers, nested primers, single specific primers, degenerate primers, gene-specific primers, vector-specific primers, partially mismatched primers, mutagenic primers, and the like. See also Innis et al., eds., PCR Protocols: A Guide to Methods and Applications (Academic Press, New York (1995); and Gelfand, eds., PCR Strategies (Academic Press, New York (1995); and Innis and Gelfand, eds., PCR Methods Manual (Academic Press, New York) (1999).

III. Transformed or Transfected Cells

The invention provides cells that have been transfected or transformed with a nucleic acid construct of the invention. The nucleic acid construct may be replicated within the cells without producing a retrovirus-like particle. The nucleic acid construct may be expressed within the cell such that retrovirus-like particles are produced.

Many types of prokaryotic and eukaryotic cells may be transfected or transformed with a nucleic acid construct of the invention. Examples of such cells include bacteria, yeast, and numerous eukaryotic cells. For example, bacterial cells may be transformed with a nucleic acid construct to provide for amplification of the nucleic acid construct. In addition, bacterial cells provide large amounts of DNA that can be readily manipulated through use of commonly known techniques. Site directed mutagenesis can be conveniently performed using DNA isolated from bacteria (Kunkel, Proc. Natl. Acad. Sci. USA, 82, 488 (1985); Kunkel et al., Methods in Enzymol., 154:367 (1987)). Additional examples of cells that can be transformed with the nucleic acid constructs of the invention include, but are not limited to, 293T human kidney cells and HeLa cells.

Methods to transform or transfect cells, and select for cells that have been transformed or transfected, are known in the art. Briefly, a nucleic acid molecule may be introduced into a cell through calcium phosphate precipitation, ballistic transformation, liposome transformation, electroporation, polyethylene glycol precipitation, and the like.

IV. Method to Increase Production of Retrovirus-Like Particles

The invention provides a method to increase production of retrovirus-like particles that involves contacting a cell that produces a retrovirus-like particle having an altered nucleocapsid domain that inhibits packaging of genomic RNA into the retrovirus-like particle, with a retroviral protease inhibitor.

It has been discovered that inhibition of retroviral protease activity increases the budding efficiency of retroviral-like particles having an altered nucleocapsid domain from the surface of a cell. Use of this method will increase production of retrovirus-like particles that can be used in the manufacture of immunologic compositions and vaccines.

The invention includes any inhibitor that decreases or eliminates the enzymatic activity of a retroviral protease from which the retroviral-protease is derived, if the inhibitor increases budding of retroviral-like particles. Examples of suitable protease inhibitors that can be used to inhibit the protease from human immunodeficiency virus include saquinavir, amprenavir, indinavir, lopinavir, nelfinavir, ritonavir, biliverdin, bilirubin, stercobilin, urobilin, biliverdin dimethyl ester, and xanthobilirubic acid. Peptide inhibitors and peptidomimetics can also be used to inhibit retroviral proteases (Blumenzweig et al., Biochem. Biophys. Res. Commun., 292:832 (2002); Beaulieu et al., J. Med. Chem., 40:2164 (1997); McPhee et al., Biochem. J., 320:681 (1996)).

Generally, the method involves culturing cells under conditions where the cells produce a retrovirus-like particles having an altered nucleocapsid that caused the retrovirus-like particles to have a reduced or an undetectable amount of genomic RNA packaged into the retrovirus-like particles. The cells are contacted with a sufficient amount of a retroviral protease inhibitor to increase production of retrovirus-like particles by the cells. It is thought that the retroviral protease inhibitor increases budding of retrovirus-like particles having altered nucleocapsid domains from cells. Methods to culture cells under conditions where they will produce retrovirus-like particles are known in the art and are described herein.

V. Immune Compositions and Vaccines of the Invention

The invention provides immune compositions and vaccines that can be used to produce an immune response against a retrovirus from which a retrovirus-like particle was prepared. For example, a retrovirus-like particle of the invention that was derived from an infectious HIV-1 clone can be used to immunize an animal, such as a human, by eliciting a protective immune response against the retrovirus-like particle that will also recognize an infectious HIV-1 virion. The immune response may be a humoral immune response or a cellular immune response.

An immune composition of the invention can include a retrovirus-like particle of the invention and a pharmaceutically acceptable diluent or carrier. An immune composition of the invention can also include an adjuvant. In some embodiments, the adjuvant is not chemically linked to the retrovirus-like particle of the invention. In other embodiments, the adjuvant is chemically linked to the retrovirus-like particle of the invention.

An immune composition may be manufactured conventionally. In particular, a retrovirus-like particle may be combined with a pharmaceutically acceptable diluent or carrier. Examples of pharmaceutically acceptable diluent or carriers include water or a saline solution, such as phosphate-buffered saline (PBS). In general, the pharmaceutically acceptable diluent or carrier is selected on the basis of the mode and route of administration and on standard pharmaceutical practices. Pharmaceutically acceptable diluents and carriers as well as all that is necessary for their use in pharmaceutical compositions are described in Remington's Pharmaceutical Sciences, a standard reference text in this field.

Immune compositions may contain adjuvants as disclosed herein and as known in the art. Aluminum compounds may be used as adjuvants. Such aluminum compounds include, aluminum hydroxide, aluminum phosphate, aluminum hydroxyphosphate, and the like. A retrovirus-like particle may be absorbed or precipitated on an aluminum compound according to standard methods. Other adjuvants include polyphosphazene (WO 95/2415), DC-chol (3-beta-[N-(N′,N′-dimethylaminomethane)carbamoyl)cholesterol] (U.S. Pat. No. 5,283,185 and WO 96/14831), QS-21 (WO 88/9336) and RIBI from ImmunoChem (Hamilton, Mont.). Immunostimulatory oligonucleotides containing unmethylated CpG dinucleotides (“CpG”) are known in the art as being adjuvants when administered by both systemic and mucosal routes (WO 96/02555, EP 468520, Davis et al., J. Immunol. 160:870 (1998); McCluskie and Davis, J. Immunol., 161:4463 (1998). CpG when formulated into immune compositions or vaccines, is generally administered in free solution together with free antigen (WO 96/02555; McCluskie and Davis, J. Immunol., 161:4463 (1998)) or covalently conjugated to an antigen (PCT Publication No. WO 98/16247), or formulated with a carrier such as aluminium hydroxide. (Brazolot-Millan et al., Proc. Natl. Acad. Sci., 95:15553 (1998)).

The invention also provides vaccines that include a retrovirus-like particle of the invention. Such vaccines can be formulated as described herein or as known in the vaccine arts. For example, vaccines can also be formulated as a liposome. Such formulations are known to those skilled in the art. Liposomes: A Practical Approach. RRC New Ed, IRL press (1990).

The invention also provides nucleic acid based vaccines that are expressed in a cell and produce a retrovirus-like particle of the invention. Inoculation of an animal or human with a nucleic acid construct of the invention may lead to a humoral and cell-mediated immune response to an infectious or non-infectious virion from which the retrovirus-like particle was derived. It is thought that some bone marrow-derived professional antigen presenting cells are transfected by the nucleic acid construct and the encoded antigens are transcribed and translated into immunogenic polypeptides that elicit specific responses. A feature of nucleic acid vaccines is that they provide for eliciting strong cytotoxic T-lymphocyte (CTL) responses. This is because the nucleic acid-encoded polypeptides are synthesized in the cytosol of transfected cells. Furthermore, nucleic acid constructs that are produced in bacteria are rich in unmethylated CpG nucleotides that are recognized as foreign by macrophages. Thus, they elicit an innate immune response that enhances adaptive immunity. Therefore, nucleic acid vaccines are effective even when administered without adjuvants.

Direct injection of a nucleic acid construct of the invention into living host cells transforms a number of the cells and causes them to express the introduced nucleic acid construct, and thereby express the gene products encoded by the nucleic acid construct. The transfected cells may display fragments of the expressed antigens on their cell surfaces together with major histocompatibility class I (MHC I) or class II (MHC II) complexes.

Nucleic acid constructs can be introduced into cells more efficiently by inducing muscle degeneration prior to the injection of the nucleic acid construct into an animal, including a human (Vitadello et. al., Hum. Gene. Ther., 5:11 (1994); Danko and Wolff, Vaccine, 12:1499 (1994); Davis et. al., Hum. Gene. Ther., 4:733 (1993)). Such treatment is thought to increase the efficiency of transfer of the nucleic acid construct by up to 40 fold. Two of the most commonly used myonecrotic agents are the local anesthetic bupivicaine, and cardiotoxin (Danko and Wolff, Vaccine, 12:1499 (1994); Davis et. al., Hum. Gene. Ther., 4:733 (1993)). A number of other techniques have been employed to transfer nucleic acid constructs to muscle. Such other techniques include viral vectors and liposomes. However, direct injection of naked nucleic acid appears to be the most efficient of these delivery mechanisms at transferring and expressing foreign nucleic acids in cells.

Nucleic acid constructs can be administered in a pharmaceutically acceptable carrier. Pharmaceutically acceptable carriers are biologically compatible vehicles which are suitable for administration to a human or other mammalian subject, e.g., physiological saline. A therapeutically effective amount is an amount of the nucleic acid construct that is capable of producing an immune response (e.g., an enhanced T-cell response or antibody production) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of a nucleic acid construct is from approximately 10⁶ to 10¹² copies of the nucleic acid construct. This does can be repeatedly administered, as needed.

Numerous routes of administration may be used to administer nucleic acid constructs. Examples of such routes include intramuscular injection, intravenous, intraperitoneal, intradermal, intranasal and subcutaneous injection of nucleic acid constructs have all resulted in immunization against influenza virus hemagglutinin (HA) in chickens (Pardoll and Beckerleg, Immunity, 3:165 (1995)). Nucleic acid based vaccines can also be administered through use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The nucleic acid construct is encapsulated in these microparticles, which are taken up by macrophages and gradually biodegraded within the cell, thereby releasing the nucleic acid construct. Once released, the nucleic acid is expressed within the cell. Another way to achieve uptake of a nucleic acid construct is through use of liposomes. Such liposomes can be prepared by standard methods. The nucleic acid constructs can be incorporated alone into these delivery vehicles or co-incorporated with tissue-specific antibodies. Alternatively, a molecular conjugate can be prepared that is composed of a nucleic acid construct attached to poly-L-lysine by electrostatic or covalent forces. Poly-L-lysine binds to a ligand that can bind to a receptor on target cells (Cristiano et al., J. Mol. Med., 73:479 (1995)). Alternatively, lymphoid tissue specific targeting can be achieved by the use of lymphoid tissue-specific transcriptional regulatory elements (TRE) such as a B lymphocyte, T lymphocyte, or dendritic cell specific TRE. Lymphoid tissue specific TRE are known (Thompson et al., Mol. Cell. Biol., 12:1043 (1992); Todd et al., J. Exp. Med., 177:1663 (1993); Penix et al., J. Exp. Med., 178:1483 (1993)).

An immune composition or vaccine may be administered by any conventional route used in the field of vaccines. For example, an immune composition or vaccine can be administered orally or by intravenous infusion, or injected subcutaneously, intramuscularly, intraperitoneally, intrarectally, intravaginally, intranasally, intragastrically, intratracheally, or intrapulmonarily. The choice of the administration route depends on a number of parameters such as the nature of the active principle; the identity of the retrovirus-like particle or nucleic acid construct; or the adjuvant that is combined with the aforementioned molecules. Administration of an immune composition may take place in a single dose or in a dose repeated once or several times over a certain period. The appropriate dosage varies according to various parameters. Such parameters include the individual treated (adult or child), the immune composition or antigen itself, the mode and frequency of administration, the presence or absence of adjuvant and, if present, the type of adjuvant and the desired effect (e.g. protection or treatment), as will be determined by persons skilled in the art.

TABLE 1

| | |
|---|---|
| virus | amino acid sequence containing arrays |

| | |
|---|---|
| FeLV | CAYCKEKGHWAKDC |
| | (SEQ ID NO.1) |
| R-MuLV | CAYCKEKGHWAKDC |
| | (SEQ ID NO.2) |
| M-MuLV | CAYCKEKGHWAKDC |
| | (SEQ ID NO.3) |
| Akv-MuLV | CAYCKEKGHWAKDC |
| | (SEQ ID NO.4) |
| BaEV | CAYCKERGHWTKDC |
| | (SEQ ID NO.5) |
| SRV-1 | CFKCGRKGHFAKNC-(X₁₅)-CPRCKRGKHWANEC |
| | (SEQ ID NO.6) |
| MMTV | CFSCGKTGHIKRDC-(X₁₃)-CPRCKKGYHWKSEC |
| | (SEQ ID NO.7) |
| RSV | CYTCGSPGHYQAQC-(X₁₂)-CELCNGMGHNAKQC |
| | (SEQ ID NO.8) |
| BLV | CYRCLKEGHWARDC-(X₁₁)-CPICKDPSHWKRDC |
| | (SEQ ID NO.9) |
| HTLV-1 | CFRCGKAGHWSRDC-(X₉)--CPLCQDPTHWKRDC |
| | (SEQ ID NO.10) |
| HIV-HXB2 | CFNCGKEGHTARNC-(X₇)--CWKCGKEGHQMKDC |
| | (SEQ ID NO.11) |
| HIV-BH102 | CFNCGKEGHTARNC-(X₇)--CWKCGKEGHQMKDC |
| | (SEQ ID NO.12) |
| HIV-PV22 | CFNCGKEGHTARNC-(X₇)--CWKCGKEGHQMKDC |
| | (SEQ ID NO.13) |
| HIV-BH5 | CFNCGKEGHIARNC-(X₇)--CWKCGKEGHQMKDC |
| | (SEQ ID NO.14) |
| HIV-BRU | CFNCGKEGHIARNC-(X₇)--CWKCGKEGHQMKDC |
| | (SEQ ID NO.15) |
| HIV-MN | CFNCGKEGHIAKNC-(X₇)--CWKCGKEGHQMKDC |
| | (SEQ ID NO.16) |
| HIV-SF2 | CFNCGKEGHIAKNC-(X₇)--CWRCGREGHQMKDC |
| | (SEQ ID NO.17) |
| HIV-CDC41 | CFNCGKEGHIARNC-(X₇)--CWKCGREGHQMKDC |
| | (SEQ ID NO.18) |
| HIV-RF | CFNCGKVGHIAKNC-(X₇)--CWKCGKEGHQMKDC |
| | (SEQ ID NO.19) |
| HIV-MAL | CFNCGKEGHLARNC-(X₇)--CWKCGKEGHQMKDC |
| | (SEQ ID NO.20) |
| HIV-ELI | CFNCGKEGHIAKNC-(X₇)--CWRCGKEGHQLKDC |
| | (SEQ ID NO.21) |
| HIV-2ROD | CWNCGKEGHSARQC-(X₇)--CWKCGKPGHIMTNC |
| | (SEQ ID NO.22) |
| HIV-2NIHZ | CWNCGKEGHSAPQW-(X₇)--CWKCGKSGHVMANC |
| | (SEQ ID NO.23) |
| SIV-MM142 | CWNCGKEGHSARQC-(X₇)--CWKCGKMDHVMAKC |
| | (SEQ ID NO.24) |
| SIV-K6W78 | CWNCGKEGHSARQC-(X₇)--CWKCGKMDHVMAKC |
| | (SEQ ID NO.25) |
| EIAV | CYNCGKPGHLSSQC-(X₅)--CFKCKQPGHFSKQC |
| | (SEQ ID NO.26) |
| VISNA | CYNCGKPGHLARQC-(X₅)--CHHCGKPGHMQKDC |
| | (SEQ ID NO.27) |

### EXAMPLES

### Example 1

### Creation of a Nucleocapsid Deletion Mutant

The pNL4-3 infectious molecular clone of HIV-1 (Adachi et al., J. Virol., 61:209 (1987)) (Genbank accession #AF324493) was altered by site-directed mutagenesis using the polymerase chain reaction (PCR)-based overlap extension procedure (Horton et al., BioTechniques, 8:528 (1990)). Briefly, SpeI-BclI or ApaI-BclI fragments containing the desired mutations were generated by the PCR procedure and cloned into pNL4-3 as previously described (Ott et al., J. Virol., 73:19 (1999)). The deletion of NC by this method fused nucleotide (nt) 1932 (the third position C of the glycine 4 codon in NC) to nt 2077 (the first position C of the glutamine 53 codon in NC).

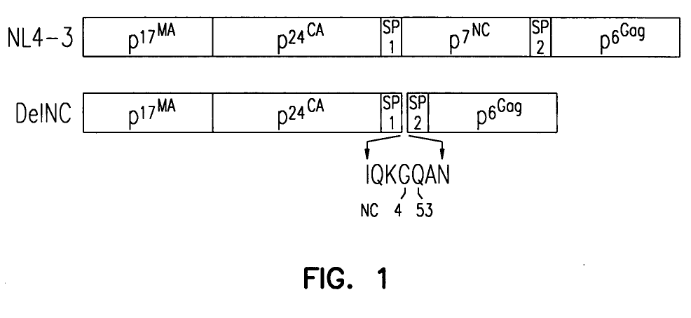

The importance of NC in HIV-1 Pr55^{Gag} was tested by removing all of the NC residues in Gag except for the first four amino acids and last three amino acids from the pNL4-3 full-length molecular clone to produce the DelNC construct (FIG. 1). The pol frameshift site and pol coding sequences were also maintained.

### Example 2

### Cell Culture Methods

The 293T transformed human kidney and HeLa-CD4-LTR-lacz (HCLZ) (gift of David Waters, AIDS Vaccine Program) cell lines were cultured in Dulbecco's modified Eagles medium; the H9 T cell leukemia line was cultured in RPMI 1640 medium. All media were supplemented with 10% vol/vol fetal bovine serum, 2 mM L-glutamine, 100 units per ml penicillin, and 100 μg per ml streptomycin. All cell culture products were obtained from Invitrogen (Carlsbad, Calif.). Transient transfections of 293T cells were carried out using the calcium phosphate method (Graham et al., Virology, 52:456 (1973)) or with 293T TransIT reagent (Mirus Corporation, Madison, Wis.) according to the manufacturer's recommendation. Virion or retroviral-particle production was measured by reverse transcriptase assay (³H-TMP incorporation using an exogenous template) on cell culture supernatants as previously described (Gorelick et al., J. Virol., 64:3207 (1990)) and CA levels were measured by an HIV-1 p24 ELISA assay (Perkin Elmer Life Sciences, Boston, Mass.). The HIV-1 infection assays using HCLZ cells as a lacz transcomplementation reporter assay for HIV-1 infection were carried out as previously described (Gorelick et al., J. Virol., 64:3207 (1990)). Virus replication assays were carried out as follows: various dilutions of virus from a transfection (approximately 6,000 cpm of reverse transcriptase activity) were used to infect 1×10 ⁶ H9 cells in a 24-well plate (Costar Corporation, Cambridge, Mass.) and clarified supernatant samples were taken periodically and monitored for reverse transcriptase activity. All HIV-1 infections were carried out in the presence of 2 μg per ml hexadimethiene bromide (polybrene, Sigma, St. Louis, Mo.).

The DelNC construct reproducibly released 6-fold less reverse transcriptase activity and 4-fold less CA into the medium upon transfection of 293T cells. The infectivity of the DelNC retrovirus-like particles produced from 293T transfections was determined by a single round assay using a Tat-transactivated lacz reporter cell line. While transfection of the pNL4-3 construct produced a titer of 1×10 ⁶ blue cell forming nits (BCFU), the DelNC construct produced a essentially no titer (4 BCFU similar to the 3 BCFU titer from the mock transfection). Similarly, the DelNC virus did not replicate on H9 cells while wild-type virus was detected at the 10⁷ dilution. Together these data show that this mutant was profoundly defective, being 10⁻⁶ to 10⁻⁷ as infectious as wild-type.

### Example 3

### Analysis of Nucleocapsid Deletion (DelNC) Retrovirus-Like Particles

Retrovirus-like particles and virions were isolated by centrifugation through a 20% sucrose pad in a SW28.1Ti rotor (Beckman-Coulter, Inc., Fullerton, Calif.) at 120,000 g at 4° C. for 1 hour. Immunoblot analyses of retrovirus-like particles and virion preparations (the equivalent of 10% of the transfection supernatant) were performed as previously described, using peroxidase-conjugated secondary antibodies and developed with the Enhanced ChemiLuminescence reagent (Amersham Life Science, Arlington Heights, Ill.). Rabbit antiserum against p6Gag (DJ-30552) and goat antiserum against, p7NC (#77), p17MA (#83), or p24CA (#81) were obtained from the AIDS Vaccine Program. Monoclonal antibody against reverse transcriptase was obtained from Perkin Elmer/NEN Life Science. N-terminal protein sequence analysis was carried out on an Applied Biosystems Procise model 494 microsequencer (Foster City, Calif.) as previously described (Chertova et al., J. Virol., 76:5315 (2002)). Micro-scale high-pressure liquid chromatography (HPLC) was performed as previously described (Chertova et al., J. Virol. 76:5315 (2002)). Matrix-assisted laser desorption/ionization-time-of-flight (MALDI) mass spectrometry was carried out as previously described (Ott et al., J. Virol., 73:19 (1999)) using a Kratos Kompact Probe (Kratos Analytical Inc., Chestnut Ridge, N.Y.).

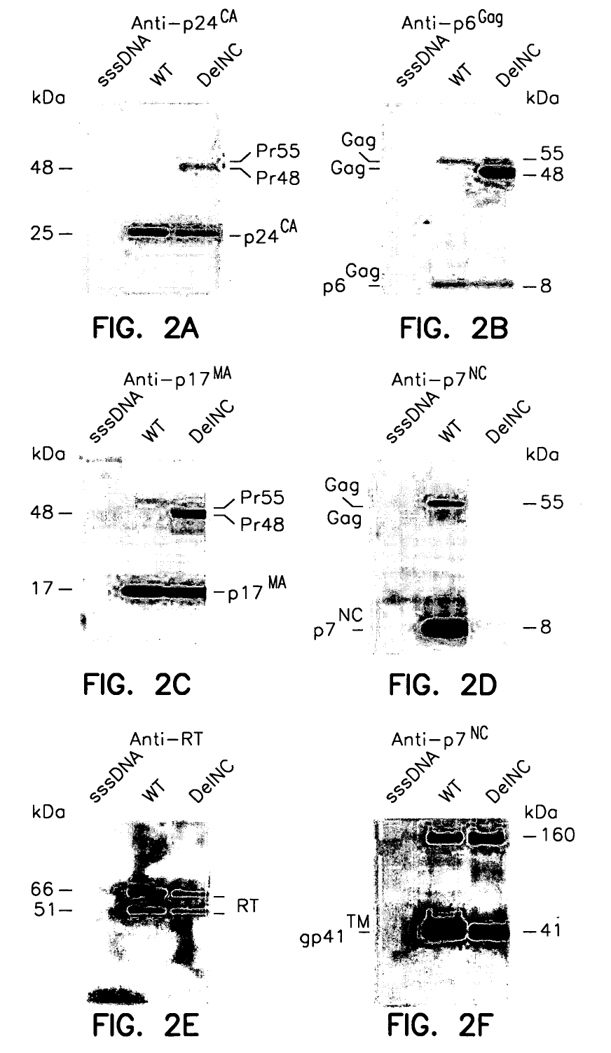

To examine the composition of the retrovirus-like particles and virions, equal percentages of the retrovirus-like particle and virions preparations produced by transfection were analyzed by immunoblotting (FIG. 2). The results with p24^{CA} antiserum showed that the DelNC construct produced a band at 25 kDa, CA, and a less intense band at 48 kDa, Gag minus NC. Compared to wild-type virions, there was somewhat more unprocessed Gag polyprotein in the DelNC retrovirus-like particles. However, there was a considerable amount of CA present in the retrovirus-like particles relative to Gag, revealing only a subtle decrease in processing. Unlike most processing defects, there was no detectable increase in Gag cleavage intermediates (FIG. 2), suggesting that DelNC Gag was either mostly processed or not cleaved at all. Immunoblotting with p6^{Gag} or p17^{MA} antiserum produced a similar result as found for wild-type: the DelNC sample contained somewhat less protein than wild-type and an increased proportion of unprocessed precursor. Stripping and reacting this blot with p7^{NC} antiserum detected NC in only the wild-type lane, no signal was detected in the DelNC sample as expected. Blotting also showed that reverse transcriptase was present in the retrovirus-like particle samples (FIG. 2), demonstrating that Pol proteins were produced and packaged into DelNC retrovirus-like particles. The presence of Env in DelNC was demonstrated by immunoblot analysis with gp41™ antibody (FIG. 2).

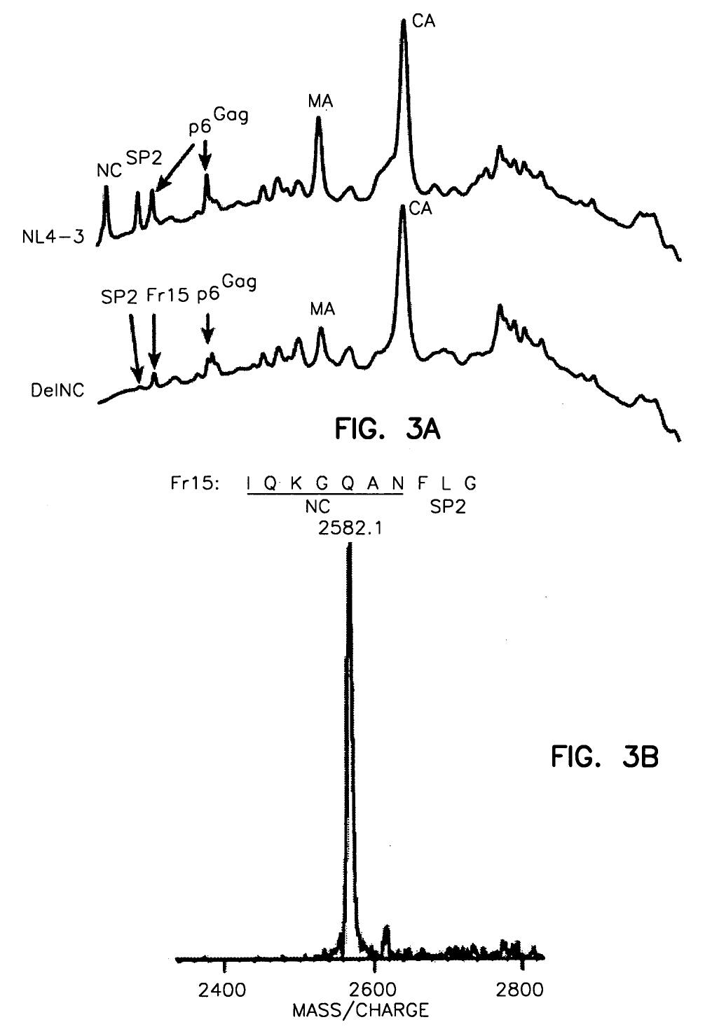

The DelNC retrovirus-like particles were further subjected to a micro-scale reversed-phase HPLC column to separate and isolate the different proteins in the retrovirus-like particles (FIG. 3A). The analysis detected all of the major mature Gag products except NC. However, protein sequence analysis of fraction 15 yielded an N-terminal sequence of a protein that matched the seven remaining residues of NC and the first three of SP2 (FIG. 3B). Mass spectroscopy showed that this protein had a molecular weight of 2581.1 kDa, consistent with the 2580.98 kDa theoretical mass for a deleted NC-SP2 protein (differing by 0.18%). Thus, DelNC retrovirus-like particles contained this partial cleavage product. Additionally, protein sequencing of DelNC fraction 14 (the fraction containing SP2 in the NL4-3 chromatogram) produced the expected SP2 N-terminal sequence: FLGXI (single amino acid code). Thus, while processing of the deleted NC-SP2 partial Gag cleavage product was not complete, it did occur. This result along with the finding that the majority of the mutant Gag protein is cleaved (FIGS. 2 and 3), shows that Gag processing is only slightly decreased.

### Example 4

### Analysis of RNA in Retrovirus-Like Particles

Northern blot analysis was performed as described previously (Gorelick et al., Virology, 253:259 (1999)) except that an 8.1 kbp Ava I fragment from pNL4-3 was used for preparing the random primed ³²P-labeled probe. The blot was washed in 0.3 M NaCl, 30 mM sodium citrate, and 0.5% (wt/vol) sodium dodceyl sulfate for 30 min at 65° C. Analysis of viral RNA by metabolic labeling was carried by transfecting 293T cells at 30% confluency, in 150 cm² flasks with TransIt reagent as before. Prior to addition of the DNA complexes, the media in the culture flasks was replaced with phosphate-free Dulbecco's modified Eagle medium (Specialty Media, Phillipsburg, N.J.) containing dialyzed fetal bovine serum (Invitrogen), 2 mM L-glutamine, and 100 unit per ml of penicillin and 100 μg per ml streptomycin. One millicurie of ³²P orthophosphoric acid, 300 Ci/mg (Perkin Elmer Life Sciences) was then added to each flask. Virions or retrovirus-like particles were collected 72 hours post-transfection by centrifugation for 1 hour at 120,000 g in an SW28.1 Ti rotor (Beckman-Coulter, Fullerton, Calif.) at 4° C. Pellets were re-suspended in lysing buffer (Gorelick et al., Virology, 253:259 (1999)) and RNA was isolated as described previously (Gorelick et al., Virology, 253:259 (1999)). RNA samples, equal to 18% of the transfection supernatant, were fractionated by formaldehyde-denaturing agarose gel electrophoresis as described above (Gorelick et al., Virology, 253:259 (1999)). The gel was dried via a gel drier onto a Whatman 3 MM filter (Clifton, N.J.) then was exposed to a phosphorimager screen (Kodak Life Sciences, Rochester, N.Y.). Images were examined using a BioRad Molecular Imager FX fluorescent imager (Hercules, Calif.) with Quantity One software for the Apple Macintosh. Real-time reverse transcriptase-PCR (RT-PCR) was carried out essentially as previously described (Suryanarayana et al., AIDS Res. Hum. Retroviruses, 14:183 (1998)) using HIV-1 specific primers and probe combination (Gorelick et al., Virology, 256:92 (1999)) that span pNL4-3 nucleotides 1367 to 1535.

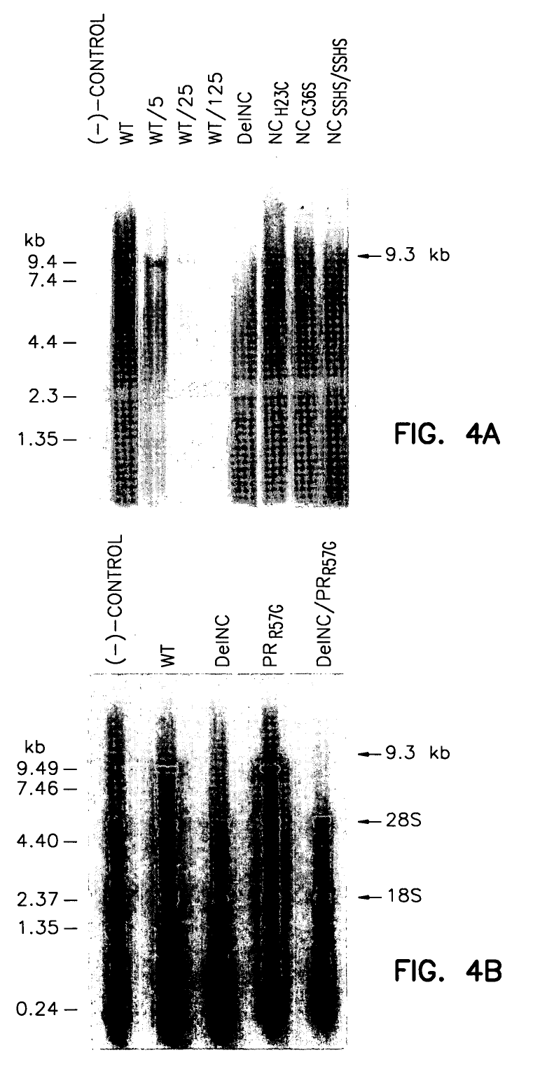

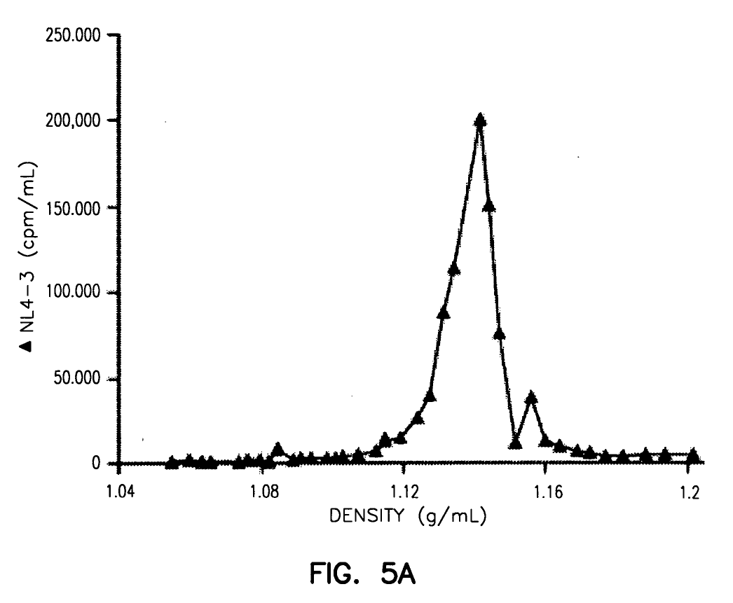

The amount of intact genomic RNA in DelNC retrovirus-like particles was examined by denaturing Northern blot analysis and was determined to be severely reduced or non-existent as compared to wild-type virions. RNA was isolated from equal amounts of retrovirus-like particles and virions as measured by reverse transcriptase activity. Samples from wild-type virions produced a band at 9.3 kb when probed with an 8.1 kb Aval HIV-1 probe, consistent with the expected size of full-length genomic RNA (FIG. 4A). However, the DelNC RNA sample did not contain any detectable genomic RNA signal. Lanes containing dilutions of the NL4-3 RNA preparation showed that a signal from a 1/125 dilution could still be detected in this blot (FIG. 4A). Therefore, the packaging of DelNC appears to be less than 0.8% of wild-type. For comparison, samples from three well-characterized Zn⁺²-finger mutants were also analyzed on this same blot. The results agreed with previously observed results: NC_{H23C}, a change in the first Zn⁺²-finger histidine to a cysteine (residue 23), packaged near wild-type (Gorelick et al., Virology, 256:92 (1999)); NC_{C36S}, a change in a second Zn⁺²-finger cysteine to a serine (residue 36), packaged between 1/5 and 1/25 as well as wild-type (Gorelick et al., J. Virol., 64:3207 (1990)); while, NC_{SSHS/SSHS}, which has the complete replacement of cysteines for serines in both Zn⁺²-fingers, failed to package detectable amounts of viral RNA (Guo et al., J. Virol., 74:8980 (2000)). To examine viral RNA content another way, ³²P-labeled RNA in virus-producing cells and retrovirus-like particle producing cells were metabolically labeled. RNA was isolated from virion preparations and retrovirus-like particle preparations, and the samples were examined by denaturing gel electrophoresis (FIG. 4B). Phosphorimaging analysis of the dried gel showed that genomic RNA was present in the wild-type NL4-3 preparation and absent in the DelNC samples, consistent with the Northern blot data. Thus, DelNC does not contain detectable amounts of intact genomic or spliced viral RNA. The presence of both 28S and 18S ribosomal RNA is most likely due to the presence of microvesicles, membrane vesicles which can contain ribosomal RNA and contaminate retrovirus-like particle preparations (Bess et al., Virology, 230:134 (1997)). However, ribosomal RNA being packaged into retrovirus-like particles cannot be exclude as ribosomes are packaged into some MuLV virions carrying certain NC mutations (Muriaux et al., J. Virol., 76:11405 (2002)).

The amount of HIV-1 RNA containing gag in virus and retrovirus-like particle preparations was quantitated by real-time RT-PCR. The results showed that the levels of gag RNA in the DelNC sample were drastically reduced from the NL4-3 sample, approximately 10,000-fold less than the wild-type sample (Table 1). To date, this is the most severe genomic RNA packaging defect for an NC mutant examined by this procedure (Gorelick et al., Virology, 253:259 (1999), Gorelick et al., Virology, 256:92 (1999)). The amount of gag detected in the DelNC sample was 1000-fold higher than a negative control (Table 1), a preparation isolated from sssDNA transfection supernatants. This residual presence of gag RNA may be due to RNA present in the retrovirus-like particle preparations within microvesicles or simply adhered to particles. The gag message does not need to be intact for this assay. The amount of particles in the DelNC sample examined was somewhat less than the wild-type (typically 4- to 6-fold lower as estimated by immunoblot) due to the assembly defect. Nonetheless, these results and those above show that DelNC has a profound defect in viral RNA packaging.

### Example 5

### Density Analysis for Nucleocapsid Deletion Mutants

Sucrose density gradient centrifugation was performed essentially as described previously (Gorelick et al., Proc. Natl. Acad. Sci. USA, 85:8420 (1988)) except that pelleted virus from four 150 cm² flasks, produced by the calcium phosphate transfection method, was applied to the gradient and 0.25 mL fractions were collected. The amounts of NL4-3 and DelNC retrovirus-like particles in the gradient fractions were monitored by reverse transcriptase assay. For gradients analyzing DelNC/PR_{R57G}, retrovirus-like particles in fractions were detected by p6Gag immunoblot and relative levels of retrovirus-like particles were quantitated by scanning densitometry of the resulting bands with the Scion Image for Windows version 4.0.2 (Scion Corp, Frederick Md.). Densities of the fractions were determined by refractive indices.

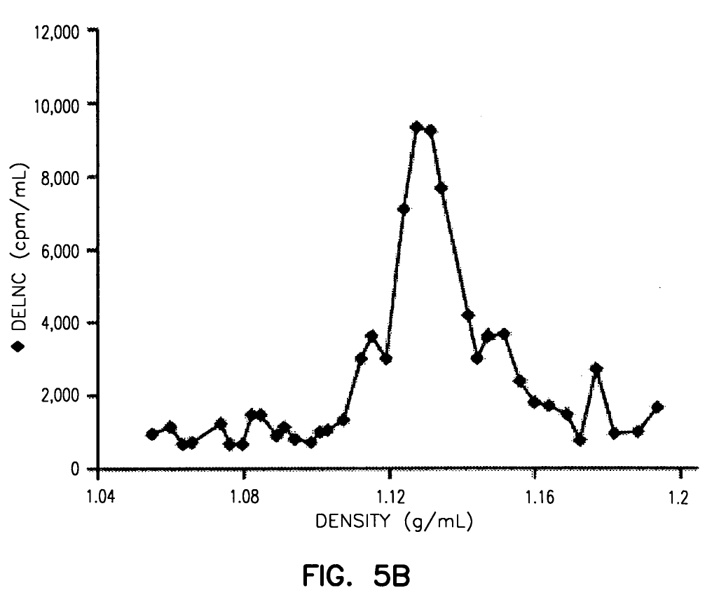

Other NC deletion mutants display a mutant I domain phenotype in that their retrovirus-like particles are less dense than wild-type, apparently due to a defect in Gag-Gag packing (Cimarelli and Luban, J. Virol., 74:6734 (2000), Dawson and Yu, Virology, 251:141 (1998), Sandefur et al., J. Virol., 74:7238 (2000), Zhang and Barklis, J. Virol., 71:6765 (1997)). To check for an I domain defect, DelNC retrovirus-like particles were examined by sucrose density gradient centrifugation. Fractions were collected, then assayed for the presence of retrovirus-like particles by reverse transcriptase activity. The results revealed that DelNC retrovirus-like particles were lighter than those from wild-type, as the mutant retrovirus-like particles banded around an average of 1.127 grams/milliliter, while the wild-type virions were distributed around 1.141 grams/milliliter (FIG. 5). The density distribution of the mutant retrovirus-like particles was wider than that of the wild-type virions, ranging from 1.11-to-1.16 grams/milliliter versus 1.13-to-1.15 grams/milliliter, respectively. The observed density shift of 0.013 grams/milliliter from the mutant to wild-type was somewhat lower than the 0.02 grams/milliliter differential measured for other I domain mutations (Bennett et al., J. Virol., 67:6487 (1993), Cimarelli and Luban, J. Virol., 74:6734 (2000), Dawson and Yu, Virology, 251:141 (1998), Sandefur et al., J. Virol., 74:7238 (2000), Zhang and Barklis, J. Virol., 71:6765 (1997)). However, this result strongly suggests a defect in the I domain of this mutant. Immunoblotting of the fractions containing peak activity confirmed the relative presence of virus in the peaks. Thus, DelNC particles are relatively lighter and more heterogeneous than those of NL4-3, characteristic of an I domain defect.

### Example 6

### Transmission Electron Microscopy

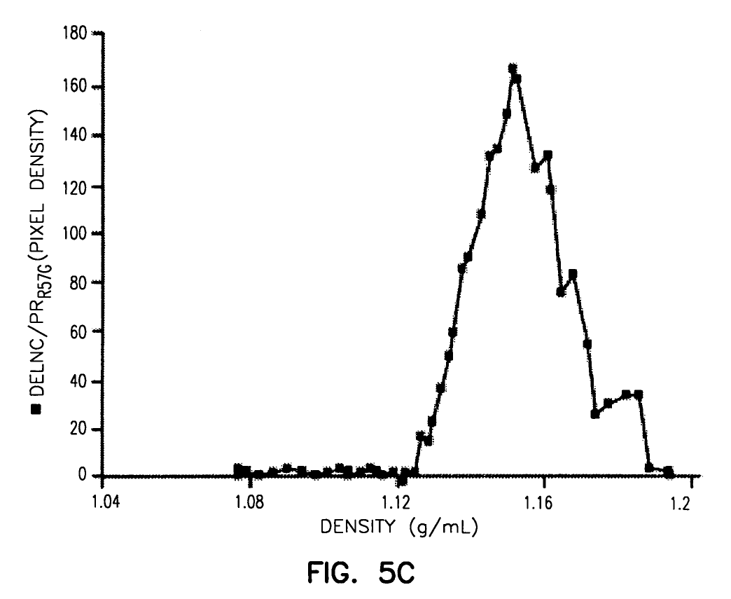

Micrographs of positive-stained virions and retrovirus-like particles were obtained as previously described (Gorelick et al., Virology, 253:259 (1999)) and indicated that DelNC retrovirus-like particles exhibit heterogeneous morphology. Preparations of NL4-3 virions and DelNC retrovirus-like particles were examined by transmission electron microscopy (FIG. 6). Unlike wild-type samples, the electron-dense staining features found in the DelNC particles were amorphous, cylindrical, or similar to the classic immature retroviral forms. In some cases, a small core-like object in an otherwise immature retrovirus-like particle morphology was seen (FIG. 6). These results show that the DelNC retrovirus-like particles were quite different from wild-type particles, possessing defects in their Gag organization. This is consistent with the density measurements presented above and confirms a defect in assembly.

### Example 7

### Creation of Protease Deficient Mutant

A protease deficient mutant, PR_{R57G}, that contained an arginine to glycine change at protease residue 57 was produced in pNL4-3 by an A-to-G change at nucleotide 2421. The Pol deficient mutant, PR_{R4X} was produced by inserting a stop codon in the pol frame at the arginine 4 of protease by introducing a C-to-T mutation at nucleotide 2274. After construction, the PCR-amplified regions of the various mutants were DNA sequenced to confirm the mutation and the integrity of the sequences exposed to the mutagenesis process.

### Example 8

### Budding of the Protease Deficient Mutant

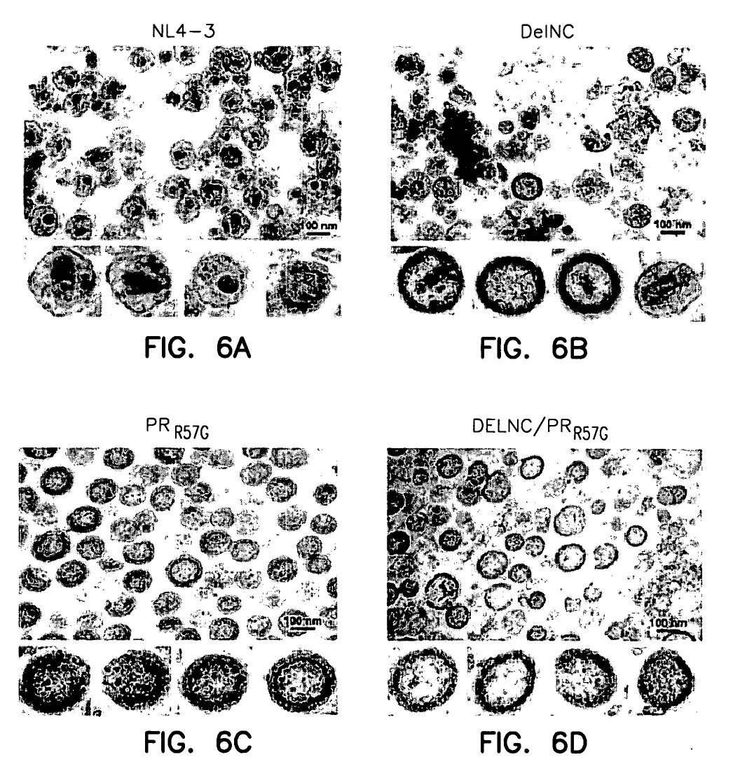

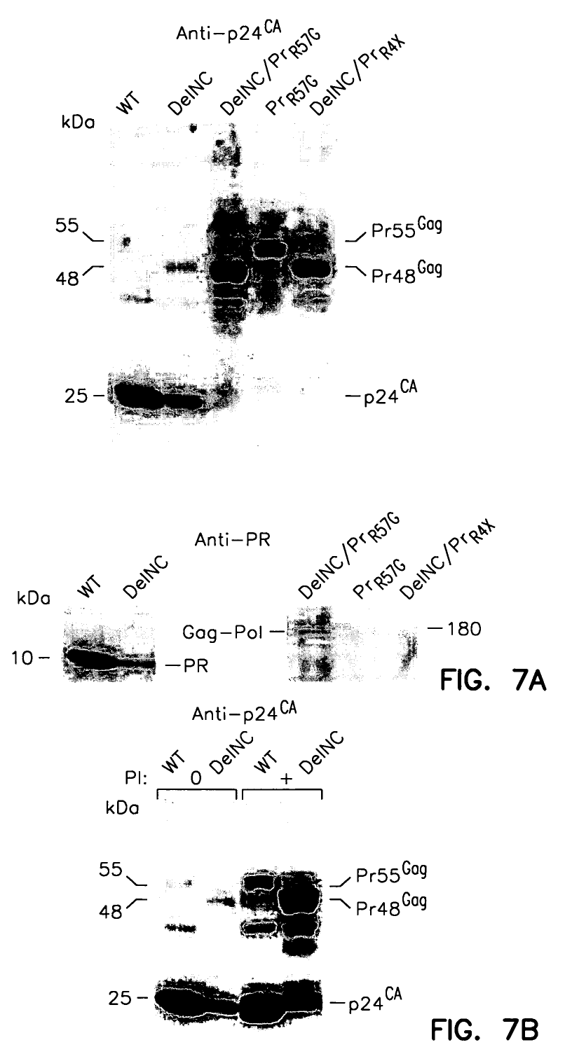

DelNC retrovirus-like particles budded inefficiently from cells. Therefore, a construct was created to determine if inactivation of the HIV protease would increase budding of retrovirus-like particles having the nucleocapsid deletion. The DelNC mutation was combined with a protease-inactivating mutation, PR_{R57G} (an arginine-to-glycine mutation at protease residue 57) to produce the DelNC/PR_{R57G} construct. Retrovirus-like particles expressed by transfection of this construct into 293T cells were examined by p24^{CA} immunoblot analysis. The results revealed that, unlike the reduced amount of CA in the DelNC versus wild-type samples, the DelNC/PR_{R57G} construct produced retrovirus-like particles at a level similar to that of the protease mutant with wild-type NC, PR_{R57G} (FIG. 7A). Stripping and reacting the blot with protease antiserum readily detected a difference in the amount of protease in the wild-type and DelNC lanes that was similar to the CA blot result. The lane containing DelNC/PR_{R57G} produced a faint yet detectable Gag-Pol while the lane containing PR_{R57G} did not (FIG. 7A). The ability to detect protease in the DelNC/PR_{57G} sample and not in that of the wild-type protease mutant demonstrates that this double mutant produces particles at least as well as the NC-containing single mutant.

### Example 9

### Examination of the Protease Activity of the Protease Inactive Mutant

To examine protease activity and mutant budding in another way, 293T cells were transfected with pNL4-3 or DelNC and then treated without or with 10 nM of the protease inhibitor Saquinavir for 24 hours. The results showed that the protease inhibitor essentially eliminated DelNC Gag processing while causing only a slight reduction in the cleavage of NL4-3 Gag at this concentration (FIG. 7B). Furthermore, the DelNC retrovirus-like particle sample produced from the protease inhibitor-treated cells contained more Gag than the corresponding material from the untreated cells. Therefore, inhibition of Gag processing increased the production of DelNC retrovirus-like particles. In contrast, the amount of pNL4-3 virions found in the medium was not largely increased by protease inhibitor (FIG. 7B), even at concentrations that blocked processing. Thus, elimination of protease activity, either by mutation or by protease inhibitor, allows the DelNC mutant to produce retrovirus-like particles efficiently. To examine any potential role that the Pol precursor itself might have in this result, a mutant was utilized that had a stop codon at position 4 of NL4-3 protease, PR_{R4X} (arginine-to-nonsense at position 4) that eliminated nearly all of Pol. A DelNC/PR_{R4X} double mutant produced similar levels of particles as the protease-deficient NC mutant as demonstrated by immunoblot (FIG. 7A). Therefore, the absence of protease activity is sufficient to rescue the DelNC assembly defect as opposed to simply the presence of uncleaved Pol.

### Example 10

### Genomic RNA Packaging in Protease Inactive Mutants

Examination of the RNA levels in DelNC/PR_{R57G} by metabolic ³²P labeling of RNA showed that similar to the results obtained with DelNC, DelNC/PR_{R57G} did not contain detectable levels of RNA (FIG. 4). Analysis of both DelNC/PR_{R57G} and DelNC/PR_{R4X} by real-time RT-PCR found that both of these double mutants had severe defects in viral RNA packaging (Table 1), approximately 10,000-fold and 1000-fold reduced from their NC-containing counterparts (i.e., PR_{R57G} and PR_{R4X}, respectively). These differences are comparable to the difference between DelNC and wild-type. Unlike the DelNC preparations, retrovirus-like particle preparations from the protease mutants and protease-DelNC mutants used in this analysis contained nearly equal amounts of retrovirus-like particles, as determined by immunoblot and HPLC. Thus rescue of assembly did not involve the restoration of genomic RNA packaging.

TABLE 1
Real-time RT-PCR analysis of virion samples

| | Number of HIV-1 | Number of copies relative |
|---|---|---|
| Virus | RNA/milliliter* | to NL4-3 |
| sssDNA | (3.49 +/− 4.93) × 10² | 2.0 × 10⁻⁷ |
| NL4-3 | (1.41 +/− 0.13) × 10⁹ | 1 |
| DelNC | (1.98 +/− 0.29) × 10⁵ | 1.4 × 10⁻⁴ |
| PR_{R57G} | (6.84 +/− 0.20) × 10⁵ | 0.49 |
| DelNC/PR_{R57G} | (2.48 +/− 0.05) × 10⁵ | 1.7 × 10⁻⁴ |
| PR_{R4X} | (2.52 +/− 0.00) × 10⁸ | 0.18 |
| DelNC/PR_{R4X} | (5.55 +/− 1.57) × 10⁵ | 3.9 × 10⁻⁴ |

*The highest level of contaminating DNA in the virion samples was 4 × 10⁻⁸ that of the number of RNA copies

### Example 11

### Morphology of Nucleocapsid Deletion/Protease Inactive (DelNC/PR_{57G}, Mutants

The retrovirus-like particles produced by DelNC/PR_{R57G} and PR_{R57G} were examined bv electron microscopy. The retrovirus-like particles produced by PR_{R57G} showed the typical distended circle or teardrop shape for protease-deficient particles (FIG. 6). These particles had no central core, rather they contained a ring of electron-dense material that covered about 270 degrees of the retrovirus-like particle with a notable thinning to absence of electron-dense material through the remaining portion of the particle. The inner border of the ring also contained a thin black band. These teardrop shapes are seen with HIV-1 Gag and Gag-Pol containing virions and not with particles formed by Gag alone (Gottlinger et al., Proc. Natl. Acad. Sci. USA, 86:5781 (1989), Jowett et al., J. Gen. Virol., 73:3079 (1992), Kaplan et al., Antimicrob. Agents Chemother., 38:2929 (1994), Schatzl et al., Arch. Virol., 120:71 (1991)), which displays the doughnut-like concentric ring morphology displayed by some other immature orthoretroviruses (Katoh et al., Virology, 145:280 (1985)). The DelNC/PR_{R57G} particles were essentially uniform in radial density even though they contained Pol proteins. Thus, they were closer in morphology to retrovirus-like particles that are produced by Gag expression (Jowett et al., J. Gen. Virol, 73:3079 (1992)). The electron-dense region just under the viral membrane in the DelNC/PR_{R57G} particles was somewhat thinner than the similar region in the PR_{R57G} particles (FIG. 6) or those produced from Gag alone (data not shown). Additionally, the inner black band was absent in these retrovirus-like particles. Despite these differences, the protease mutation in DelNC/PR_{R57G} reversed the morphologically observed assembly defect of the DelNC mutation, allowing for the production of uniform particles.

DelNC/PR_{R57G} forms dense retrovirus-like particles. The density of DelNC/PR_{R57G} was measured by sucrose gradient centrifugation and the fractions examined for the presence of Pr48Gag by immunoblotting. The relative amounts of Gag signal on the blot were measured by pixel grayscale density and used to quantitate the relative amount of DelNC/PR_{R57G} in the gradient fractions (FIG. 5). The results showed that the peak of virus recovery was at approximately 1.151 grams/milliliter, considerably denser than the DelNC particles and slightly heavier than wild-type. Therefore, the absence of protease activity appears to have restored the I domain function to the DelNC/PR_{R57G} mutant.

All publications, patents and patent applications cited herein are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

What is claimed is:

1. A retrovirus-like particle comprising an altered nucleocapsid domain that inhibits packaging of genomic RNA into the retrovirus-like particle, and a protease having reduced enzymatic activity, wherein the altered nucleocapsid domain has a mutation in a cysteine array having an amino acid sequence
(SEQ ID NO:28)
-Cys-X-X-Cys-X-X-X-X-His-X-X-X-X-Cys-,
where X represents a variable amino acid.

2. The retrovirus-like particle according to claim 1, wherein at least one cysteine or histidine in the cysteine array is exchanged with another amino acid or is deleted, or wherein the histidine and at least one cysteine in the cysteine array is exchanged with another amino acid or deleted.

3. The retrovirus-like particle according to claim 1, wherein at least two cysteines in the cysteine array are exchanged with another amino acid.

4. The retrovirus-like particle according to claim 1, wherein at least one amino acid represented by X is exchanged with another amino acid or is deleted.

5. The retrovirus-like particle according to claim 1, wherein at least a portion of the nucleocapsid domain has been deleted.

6. The retrovirus-like particle according to claim 1, wherein the retrovirus-like particle is derived from a pNL4-3 molecular clone of HIV-1 in which amino acids 5-52 of the nucleocapsid domain have been deleted.

7. The retrovirus-like particle according to claim 1, wherein the retroviral protease enzymatic activity is reduced by at least one order of magnitude when compared to a wild-type corresponding protease.

8. The retrovirus-like particle according to claim 1, wherein the protease lacks detectable enzymatic activity.

9. The retrovirus-like particle according to claim 1, wherein the retrovirus-like particle is derived from a pNL4-3 molecular clone of HIV-1 in which amino acid 57 of the protease has been substituted with another amino acid.

10. The retrovirus-like particle according to claim 9, wherein the protease is inactivated by exchange of arginine at amino acid position 57 with glycine.

11. The retrovirus-like particle according to claim 1, wherein an aspartic acid at amino acid position 25 in the protease has been substituted with another amino acid.

12. The retrovirus-like particle according to claim 1, wherein the protease is inactivated by deletion of a portion of the protease.

13. The retrovirus-like particle according to claim 1, wherein the infectivity of the retrovirus-like particle is reduced by at least three to five orders of magnitude when compared to a wild-type virion.

14. The retrovirus-like particle according to claim 1, wherein the retrovirus-like particle is non-infectious.

15. The retrovirus-like particle according to claim 1, wherein genomic RNA content in the retrovirus-like particle is reduced by at least one order of magnitude when compared to a wild-type virion.

16. The retrovirus-like particle according to claim 1, wherein genomic RNA content in the retrovirus-like particle is reduced by at least three to five orders of magnitude when compared to a wild-type virion.

17. The retrovirus-like particle according to claim 1, wherein genomic RNA is not detectable in the retrovirus-like particle.

18. The retrovirus-like particle according to claim 1, wherein the retrovirus-like particle is derived by mutating a human immunodeficiency virus.

19. The retrovirus-like particle according to claim 1, wherein the retrovirus-like particle is derived by mutating a pNL4-3 infectious molecular clone having GenBank accession number AF324493.

20. A nucleic acid construct comprising a full-length proviral clone that encodes a retrovirus-like particle having an altered nucleocapsid domain that inhibits packaging of genomic RNA into the retrovirus-like particle, and a protease having reduced enzymatic activity when expressed in a host cell, wherein the altered nucleocapsid domain has a mutation in a cysteine array having an amino acid sequence
(SEQ ID NO:28)
-Cys-X-X-Cys-X-X-X-X-His-X-X-X-X-Cys-,
where X represents a variable amino acid.

21. The nucleic acid construct according to claim 20, wherein at least one cysteine or histidine in the cysteine array is exchanged with another amino acid or is deleted.

22. The nucleic acid construct according to claim 20, wherein the histidine and at least one cysteine in the cysteine array is exchanged with another amino acid.

23. The nucleic acid construct according to claim 20, wherein at least two cysteines in the cysteine array are exchanged with another amino acid.

24. The nucleic acid construct according to claim 20, wherein at least one amino acid represented by X is exchanged with another amino acid or is deleted.

25. The nucleic acid construct according to claim 20, wherein at least a portion of the nucleocapsid domain has been deleted.

26. The nucleic acid construct according to claim 20, wherein the nucleic acid construct is derived from a pNL4-3 molecular clone of HIV-1 in which nucleic acid sequence encoding amino acids 5-52 of the nucleocapsid domain has been deleted.

27. The nucleic acid construct according to claim 20, wherein nucleic acid sequence encoding the nucleocapsid domain has been deleted.

28. The nucleic acid construct according to claim 20, wherein the nucleic acid construct is derived from a pNL4-3 molecular clone of HIV-1 in which nucleic acid sequence encoding amino acid 57 of the protease has been mutated to code for a different amino acid.

29. The nucleic acid construct according to claim 28, wherein the nucleic acid sequence encoding the protease is mutated to code for glycine at amino acid position 57 instead of arginine.

30. The nucleic acid construct according to claim 20, wherein nucleic acid sequence encoding the protease is mutated so that a portion of the protease is deleted.

31. The nucleic acid construct according to claim 20, wherein the construct is derived by mutating a nucleic acid sequence that codes for human immunodeficiency virus.

32. The nucleic acid construct according to claim 20, wherein the construct is derived by mutating a pNL4-3 infectious molecular clone having GenBank accession number AF324493.

33. A prokaryotic or eukaryotic cell comprising the nucleic acid construct according to claim 20.

34. A method to increase production of retrovirus-like particle comprising contacting a host cell that produces a retrovirus-like particle having an altered nucleocapsid domain that inhibits packaging of genomic RNA into the retrovirus-like particle with a retroviral protease inhibitor.

35. The method according to claim 34, wherein the retroviral protease inhibitor is saquinavir, amprenavir, indinavir, lopinavir, nelfinavir, or ritonavir.

36. The method according to claim 34, wherein the retrovirus like-particle is derived from a retrovirus.

37. The method according to claim 36, wherein the retrovirus is human immunodeficiency virus.

38. A retrovirus-like particle produced according to the method of claim 34.

39. A method of producing a retrovirus-like particle having a deletion in the nucleocapsid region and an inactivated retroviral protease comprising:
expressing a nucleic acid construct encoding the retrovirus-like particle in a host cell for a sufficient time to produce the retrovirus-like particle, and
isolating the retrovirus-like particle.

40. The method according to claim 39, wherein the host cell is contacted with a retroviral protease inhibitor.

41. The method according to claim 40, wherein the retroviral protease inhibitor is saquinavir, amprenavir, indinavir, lopinavir, nelfinavir, or ritonavir.

42. The method according to claim 39, wherein the retrovirus like-particle is derived from an infectious retrovirus.

43. The method according to claim 42, wherein the infectious retrovirus is human immunodeficiency virus.

44. An immunogenic composition comprising an isolated retrovirus-like particle having an altered nucleocapsid domain that inhibits packaging of genomic RNA into the retrovirus-like particle, and a protease having reduced enzymatic activity, wherein the nucleocapsid domain and the protease are derived from a retrovirus, and a pharmaceutically acceptable carrier.

45. The immunogenic composition according to claim 44, wherein the retroviral like-particle is derived from an infectious retrovirus.

46. The immunogenic composition according to claim 45, wherein the infectious retrovirus is human immunodeficiency virus.

47. The immunogenic composition according to claim 44, further comprising an adjuvant.

48. The immunogenic composition according to claim 47, wherein the adjuvant is selected from the group consisting of aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazine, a biodegradable microsphere, a monophosphoryl lipid A, and quil A.

49. A vaccine comprising the immunogenic composition of claim 44.

50. An immunogenic composition comprising a nucleic acid construct that encodes a retrovirus-like particle having an altered nucleocapsid domain that inhibits packaging of genomic RNA into the retrovirus-like particle, and a protease having reduced enzymatic activity, wherein the nucleocapsid domain and the protease are derived from a retrovirus; and a pharmaceutical carrier.

51. The immunogenic composition according to claim 50, wherein the composition is formulated in unit dosage form.

52. The immunogenic composition according to claim 50, wherein the composition further comprises a myonecrotic agent.

53. The immunogenic composition according to claim 52, wherein the myonecrotic agent is bupivicaine or cardiotoxin.

54. The immunogenic composition according to claim 50, further comprising an adjuvant.

55. The immunogenic composition according to claim 54, wherein the adjuvant is selected from the group consisting of aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazine, a biodegradable microsphere, a monophosphoryl lipid A, and quil A.

56. The immunogenic composition according to claim 50, formulated as a DNA vaccine.

57. A method to immunize a mammal against an infectious retrovirus comprising administering a therapeutically effective amount of an immunogenic composition according to claim 44 to the mammal in need thereof.

58. A method to immunize a mammal against an infectious retrovirus comprising administering a therapeutically effective amount of an immunogenic composition according to claim 50 to the mammal in need thereof.

59. A non-infectious mutant of an infectious retrovirus comprising a form of the infectious retrovirus having a mutation or deletion in a nucleocapsid domain of the infectious retrovirus, and a mutation or deletion in a protease domain of the infectious retrovirus, wherein the mutation or deletion is present in a cysteine array present in the nucleocapsid domain of the infectious retrovirus, the cysteine array having the sequence
(SEQ ID NO:28)
-Cys-X-X-Cys-X-X-X-X-His-X-X-X-X-Cys-,
wherein X represents variable amino acids

60. The non-infectious mutant according to claim 59, wherein the infectious retrovirus is human immunodeficiency virus.

61. A kit comprising packaging material and an immunogenic composition according to claim 44.

62. A kit comprising packaging material and an immunogenic composition according to claim 50.
